(19) United States
(12) Patent Application Publication
(10) Pub. No.: US 2023/0248862 A1
(43) Pub. Date: Aug. 10, 2023

(54) LIGHT DISINFECTION SYSTEM CONTROL SYSTEMS

(71) Applicant: Current Lighting Solutions, LLC, East Cleveland, OH (US)

(72) Inventors: Kevin J. Benner, Solon, OH (US); Gary R. Allen, Euclid, OH (US); Stephen P. Glaudel, Hatfield, PA (US)

(73) Assignee: Current Lighting Solutions, LLC, East Cleveland, OH (US)

(21) Appl. No.: 18/013,384

(22) PCT Filed: Nov. 12, 2020
(86) PCT No.: PCT/US2020/060132

§ 371 Date: Dec. 28, 2022

Related U.S. Application Data
(60) Provisional application No. 63/047,722, filed on Jul. 2, 2020.
(60) Provisional application No. 63/054,382, filed on Jul. 21, 2020.
(60) Provisional application No. 63/073,568, filed on Sep. 2, 2020.

(51) Int. Cl. A61L 2/10; A61L 2/24; A61L 9/20

(52) U.S. Cl. CPC A61L 2/10; A61L 2/24; A61L 9/20; A61L 2202/11; A61L 2202/14; A61L 2202/25; A61L 2209/12

(57) ABSTRACT

A disinfection system includes one or more light sources configured to emit ultraviolet light effective for inactivating pathogens in an environment for human occupancy, and one or more occupancy sensors configured to acquire data indicative of occupancy of the environment for human occupancy. Intensity of the ultraviolet light emitted by the one or more light sources is controlled based on the data indicative of occupancy of the environment acquired by the one or more occupancy sensors. In another embodiment, a light source for disinfection includes an intensity setting input disposed on the light source. The intensity setting input is operative to set an intensity of light emitted by light emitting elements of the light source. The light source has no other control besides the intensity setting input.

This application claims the benefit of U.S. Provisional Application No. 63/073,568 filed Sep. 2, 2020 and titled “LIGHT DISINFECTION SYSTEM CONTROL SYSTEMS”. This application claims the benefit of U.S. Provisional Application No. 63/054,382 filed Jul. 21, 2020 and titled “MULTISPECTRAL LIGHT DISINFECTION SYSTEM AND METHOD”. This application claims the benefit of U.S. Provisional Application No. 63/047,722 filed Jul. 2, 2020 and titled “LIGHT DISINFECTION SYSTEM AND METHOD”. U.S. Provisional Application No. 63/073,568 filed Sep. 2, 2020 is incorporated herein by reference in its entirety. U.S. Provisional Application No. 63/054,382 filed Jul. 21, 2020 is incorporated herein by reference in its entirety. U.S. Provisional Application No. 63/047,722 filed Jul. 2, 2020 is incorporated herein by reference in its entirety.

### BACKGROUND

The following relates to the disinfection arts, pathogen control arts, viral pathogen control arts, lighting arts, and the like.

Clynne et al., U.S. Pat. No. 9,937,274 B2 issued Apr. 10, 2018 and Clynne et al., U.S. Pat. No. 9,981,052 B2 (which is a continuation of U.S. Pat. No. 9,937,274) provide, in some illustrative examples, disinfection systems that includes a light source configured to generate ultraviolet light toward one or more surfaces or materials to inactivate one or more pathogens on the one or more surfaces or materials. U.S. Pub. No. 2016/0271281 A1 is the published application corresponding to U.S. Pat. No. 9,937,274. U.S. Pub. No. 2016/0271281 A1 is incorporated herein by reference in its entirety to provide general information on disinfection systems for occupied spaces that use ultraviolet light.

Moreno, “Effects on illumination uniformity due to dilution on arrays of LEDs”, 2004 Proceedings of SPIE, provides an approach for computing the spatial distribution of irradiance from a light emitting diode (LED) on a plane illuminated by the LED.

Wladyslaw Kowalski, ULTRAVIOLET GERMICIDAL IRRADIATION HANDBOOK (Springer-Verlag Berlin Heidelberg 2009) (hereinafter “Kowalski 2009”) provides information for estimating rate constants for inactivation of pathogens.

Certain improvements are disclosed.

### BRIEF DESCRIPTION

In some illustrative embodiments disclosed herein, a disinfection system includes one or more light sources configured to emit ultraviolet light effective for inactivating pathogens in an environment for human occupancy, and one or more sensors configured to acquire data indicative of the environment for human occupancy. The intensity and/or spectrum of the ultraviolet light emitted by the one or more light sources is controlled based on the data indicative of the environment acquired by the one or more sensors.

In some illustrative embodiments disclosed herein, a method is disclosed of configuring a light source to be used for disinfecting an environment for human occupancy. A light source-to-head level distance for the environment is determined. A maximum permissible irradiance for safe occupation is determined based on a dose time period and an actinic dose limit. A light source intensity for the light source is determined based on the light source-to-head level distance and the maximum permissible irradiance for safe occupation. An intensity setting input of the light source is operated to adjust an intensity output by the light source to the determined irradiance.

In some illustrative embodiments disclosed herein, a light source for disinfection includes an ultraviolet light source configured to emit ultraviolet light and an intensity setting input disposed on the light source for disinfection. The intensity setting input is operative to set an intensity of the ultraviolet light emitted by the ultraviolet light source. The light source for disinfection has no other control besides the intensity setting input.

In some illustrative embodiments disclosed herein, a light source for disinfection includes an ultraviolet light source configured to emit ultraviolet light, a clock, and electronics configured to control an intensity and/or spectrum of the ultraviolet light emitted by the ultraviolet light source based on a date and/or time provided by the clock.

In some illustrative embodiments disclosed herein, a disinfection system comprises: one or more light sources configured to emit light including at least ultraviolet light effective for inactivating pathogens in an environment for human occupancy; one or more sensors configured to acquire data indicative of the environment for human occupancy; and electronics included or operatively connected with the one or more light sources and configured to control an intensity and/or spectrum of the ultraviolet light emitted by the one or more light sources based on the data indicative of the environment acquired by the one or more sensors.

In some illustrative embodiments disclosed herein, a disinfection system comprises: a plurality of light sources distributed in an environment for human occupancy and configured to emit light including at least ultraviolet light; sensors distributed in the environment for human occupancy and configured to acquire data indicative of real-time spatially resolved occupancy of the environment for human occupancy; and at least one electronic processor operatively connected with the light sources and the sensors. The at least one electronic processor is programmed to: generate an occupancy map of the environment for human occupancy using the data indicative of real-time spatially resolved occupancy; determine intensities for respective light sources of the plurality of light sources based on the occupancy map and locations of the light sources of the plurality of light sources in the occupancy map; and control the respective light sources of the plurality of light sources to emit light at the intensity determined for that light source. In some embodiments, the determination of the intensities for the respective light sources includes determining a high intensity for light sources that do not impinge on an occupant as indicated by the occupancy map, where the high intensity exceeds an intensity that would produce a dose exceeding an actinic dose limit if received over a design-basis dose time period (e.g. 24 hours) over which the actinic dose limit is defined.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 diagrammatically illustrates a viral disinfection system is configured to disinfect an environment for human occupancy.

FIG. 2 diagrammatically illustrates an embodiment of a light source of the viral disinfection system of FIG. 1 which employs light emitting diodes (LEDs).

FIG. 3 diagrammatically illustrates an embodiment of a light source of the viral disinfection system of FIG. 1 which employs a mercury lamp.

FIG. 4 diagrammatically illustrates a viral disinfection method suitably performed using the viral disinfection system of FIG. 1.

FIGS. 5 and 6 illustrate two respective methods for controlling the light emitted by the light sources of the system of FIG. 1 based on occupancy as indicated by a motion sensor (FIG. 5) or microphone (FIG. 6).

FIG. 7 illustrates a control method employing a proximity or distance sensor to provide a failsafe interlock against a person coming too close to a light source that is emitting potentially hazardous ultraviolet light.

FIGS. 8-12 diagrammatically show various embodiments of multispectral light sources for disinfection as described herein.

FIG. 13 diagrammatically illustrates a multispectral light source for disinfection that is programmable to implement a spectrum tailored for disinfecting a specific target pathogen.

FIG. 14 diagrammatically illustrates a control method for controlling disinfecting ultraviolet light emitted into an environment for human occupancy based on time and/or date.

FIG. 15 diagrammatically illustrates a control method for controlling disinfecting ultraviolet light emitted into an environment for human occupancy with spatial granularity.

FIGS. 16 and 17 diagrammatically illustrate control methods for controlling disinfecting UVA light emitted into an environment for human occupancy in a manner that suppresses photoreactivation of bacteria inactivated by the UVA light.

FIG. 18 diagrammatically illustrates a viral disinfection system is configured to disinfect an environment for human occupancy, which includes multiple ways of controlling the ultraviolet light emission.

### DETAILED DESCRIPTION

Ultraviolet (UV) radiation or UV light pertains to the range between 100 nm and 400 nm, commonly subdivided into UVA, from 320 nm to 400 nm (or 315 nm to 400 nm as per some regulatory bodies such as the Illuminating Engineering Society, IES); UVB, from 280 nm to 320 nm (or 280 nm to 315 nm per the IES); and UVC, from 100 nm to 280 nm. Violet light pertains to light in the range between 380 and 450 nm. Visible light is sometimes designated as the range 380-750 nm, although the precise boundary of human visual perception varies near (and in some persons beyond) the endpoints of this range. As used herein, the term “light” encompasses visible light and also UV light and near infrared (IR) light (sometimes designated as the wavelength range 750 nm to 1400 nm).

The wavelength of a narrow-band light source, such as an LED or laser diode is understood to mean the peak wavelength, even though light is emitted from a narrow band of wavelengths shorter and longer than the peak wavelength, e.g. the full-width at half-maximum of an LED may be about 10 nm, or about +/−5 nm around the peak wavelength, with some emission even outside of the +/−5 nm range.

The peak wavelength of a narrow-band light source is understood to mean the wavelength having the highest spectral power [W/nm] of any wavelength in the emission spectrum of the light source.

The peak wavelength of a broad-band light source, or a light source having more than one emission line or band, such as a discharge lamp or excimer lamp is also understood to mean the wavelength having the highest spectral power [W/nm] of any wavelength in the emission spectrum of the light source.

The actinic dose (e.g., [J/m²]) is the quantity obtained by weighting spectrally the spectral dose of light according to the actinic action spectrum. One suitable actinic action spectrum is the published ACGIH 62471 action spectrum.

The actinic dose exposure limit for exposure to ultraviolet radiation incident upon the unprotected skin or eye apply to exposure within a defining dose time period, which is typically any 24-hour period. To protect against injury of the eye or skin from ultraviolet radiation exposure produced by a broadband source, the effective integrated spectral irradiance (effective radiant exposure, or effective dose), Eₛ, of the light source over the dose time period should not exceed 30 J/m². The effective integrated spectral irradiance, Eₛ, is defined as the quantity obtained by weighting spectrally the dose (radiant exposure) over the dose time period according to the actinic action spectrum value at the corresponding wavelength.

An environment for human occupancy is an environment that is expected to be occupied by persons (even if it is not occupied at a given time). By way of nonlimiting illustrative example, an environment for human occupancy can be an indoor environment such as a room (which could be a conference room, medical operating room, a hallway, or so forth); or can be a vehicle interior, e.g. an automobile interior, truck interior, an aircraft cabin, a spacecraft interior, a train compartment, or so forth. In these various embodiments, the environment for human occupancy has a floor, such as the floor of the room, the floor of the vehicle or aircraft cabin, or the floor of the train compartment. More generally, an environment for human occupancy can be any space that does not receive a UV component of sunlight exceeding the maximum allowed actinic exposure, for example an outdoor space having a ceiling that at least partially blocks direct sunshine, for example a storage area for shopping carts, a carport, a tunnel, a cave, a mine, an amphitheater, a stadium, a dugout, a lean-to, a tarp or tent, a picnic pavilion, or another structure not necessarily classified as a building, but which may be occupied by humans, or any outdoor area that is not instantaneously receiving a UV component of sunlight exceeding the maximum allowed actinic exposure, for example a cloudy or nighttime or seasonally dark environment. In such an environment that does not receive a UV component of sunlight exceeding the maximum allowed actinic exposure, an electric light source of this disclosure may provide UV up to the maximum allowed actinic limit, in combination with any possible sunlight contribution to the actinic limit.

As described in U.S. Pub. No. 2016/0271281 A1, UVA light at nonhazardous levels is effective for inactivating certain pathogens in an environment for human occupancy at dose levels acceptable in an occupied space, especially bacterial pathogens. A single bacterium typically has a size of about 1-10 microns in diameter or length. UVA is typically efficacious in inactivating bacteria by depositing its energy in the outer membrane of the cell, or the cell wall, where the energy of the UVA photon is sufficient to create reactive oxygen species (ROS) or to drive other chemical reactions that may cause enough damage to the cell envelope to kill or inactivate the bacterium. Violet light can also be effective for inactivating certain pathogens, especially bacterial pathogens. However, light at these longer wavelengths tends to be less effective for inactivating virus pathogens.

On the other hand, UVC light is effective for inactivating certain pathogens in an environment for human occupancy at dose levels acceptable in an occupied space, especially virus pathogens at dose levels acceptable in an occupied space. A typical virus is small, e.g. well under 1 micron in diameter or length in many cases. By way of illustration, a single coronavirus particle has a size of about 0.1 micron in diameter. As a result, UVC radiation can penetrate the outside capsid or protective layer of a virus and damage the nucleic acid contained inside a virus particle very rapidly, while it's suspended in air, e.g. in less than eight hours, or less than about 1 to 3 hours, or less than about 10 to 30 minutes, with a time-accumulated dose of about 10 J/m² or less (the dose at 254 nm required to inactive about 90% of a typical virus in air). However, at the levels of Irradiance that allow for human occupancy (e.g. below the ACGIH Actinic limit), UVC radiation often may not penetrate a bacterium with sufficient dose to damage the nucleic acids of the bacterium sufficiently to inactivate a broad range of species of bacteria (although it can kill certain common bacterial pathogens, such as E. coli). Hence, UVC light tends to be less effective for inactivating the broadest range of bacterial pathogens than is UVA. However, the combination of those two wavelengths (UVC+UVA) can provide the optimal ‘pathogen kill’ (Viricidal plus Bactericidal) in many use-cases.

Moreover, irradiance also impacts efficacy of UVA, UVC, violet, or other light in inactivating pathogens. For a given pathogen and light wavelength or spectrum, higher intensity is generally more effective than lower intensity for pathogen inactivation. However, if the light source is in use in an occupied (or possibly occupied) space, a counter-constraint is to keep the actinic dose below the actinic dose exposure limit so as to ensure safety of any human occupants.

Still further, time of exposure and even time sequence of exposure can further impact efficacy of UVA, UVC, violet, or other light in inactivating pathogens. For example, some bacteria can repair genetic damage caused by ultraviolet light by action of photolyases or other DNA repair enzymes, a process known as photoreactivation. However, photolyases are themselves light activated, typically by light in the blue or violet spectral range. Hence, an optimal disinfection sequence for such bacteria may include applying UVA light to inactivate the bacteria followed by a period of darkness to prevent photoreactivation of the damaged bacterial DNA.

As another example, inactivation of some pathogens is dependent on intensity; whereas, the actinic dose exposure limit is dose-dependent rather than instantaneous intensity-dependent. Hence, it may be beneficial to employ a pulsed UV light source for disinfection, so as to have time intervals of higher intensity than the average intensity to promote pathogen inactivation, and time intervals of lower or zero intensity to reduce the time-integrated intensity (i.e. dose), also known as the Time-Weighted Average (TWA) dose, over the dose time period for which the actinic dose exposure limit is defined.

Time and/or intensity of exposure may even further be advantageously adjusted for other reasons, such as to temporarily increase inactivation of an airborne pathogen in response to a detected cough or loud speaking or physical activity or other detected human activity that may be actively producing airborne pathogens.

An even further consideration is that various of the foregoing principles may not necessarily hold universally for all types of pathogens. In general, a given pathogen (specific species or class of virus, bacteria, or other type of pathogen such as fungi) will typically be most effectively inactivated by a particular UV (and possibly violet) light spectrum and/or particular time sequence. That is, the optimal light spectrum and/or time sequence is in general pathogen species- or class-dependent. The optimal spectrum and/or time sequence for a pathogen of interest (e.g., a pathogen that is currently causing a pandemic or local or seasonal illnesses) can be determined experimentally. Hence, in some embodiments, light source controls are provided to set the light source output spectrum and/or time sequence. These controls are preferably adjustable automatically, by the user, and/or by the manufacturer on a seasonal or other time basis to optimally disinfect different types of pathogens as they become (potentially) prevalent in the environment.

Further considerations can be relevant if the light source(s) used in disinfection are capable of outputting a light dose exceeding the actinic dose exposure limit. Various safety interlocks or other safety features may be incorporated in such a light source. In one approach, the manufacturer presets the controls (i.e. pre-configures the light source) for a specific end-use case, and these preset controls are not adjustable by the user. In another scenario, sensors may be included that detect situations in which the actinic dose exposure limit must not be exceeded, e.g. occupancy sensors may be included and the light output intensity controlled on the basis of whether the environment is occupied (for example, outputting at intensity that will exceed the actinic dose limit over the dose time period only when the environment is unoccupied). If the light source is sold in a retail setting, it should be designed to automatically ensure such safety enforcement. A difficulty here is that the actinic dose exposure limit depends on factors beyond the characteristics of the light source itself, such as its placement in the environment for human occupancy and the number and locations of the light sources.

Various control systems are disclosed herein which address the foregoing situations and others.

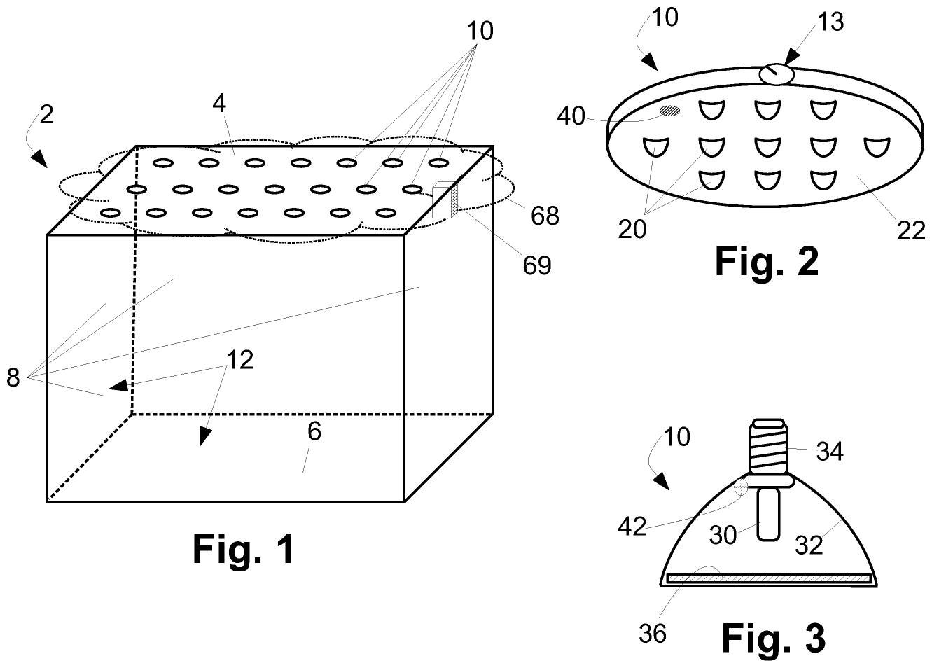

With reference to FIG. 1, a disinfection system is configured to disinfect an environment 2 for human occupancy, such as the room 2 having a ceiling 4, floor 6, and walls 8 that is occupied by persons. More generally, the environment 2 for human occupancy can be a room (which could be a conference room, medical operating room, a hallway, or so forth), or a vehicle cabin, an aircraft cabin, train compartment, or so forth, or even an outdoor environment (which could be a shopping cart corral or picnic venue, or so forth). In these various embodiments, the environment 2 for human occupancy has a floor 6, such as the illustrative floor 6 of the room, the floor of the vehicle or aircraft cabin, or the floor of the train compartment. In the case of an outdoor environment, the floor 6 is considered the ground of the outdoor environment. It will be appreciated that the portion of the environment 2 that is actually occupied by persons is typically the space that is approximately two meters or closer (e.g. 2.1 meters or closer in some embodiments) to the floor 6, which is the expected occupancy in a normal work environment. Hence, the disinfection system is typically designed to provide disinfection at a target plane, where the target plane is two meters or closer to the floor 6. The disinfection system includes at least one light source 10 configured to emit light into the environment 2 for human occupancy to inactivate one or more pathogens suspended in ambient air of the environment 2 or residing on surfaces 12 or materials, including human skin. The illustrative at least one light source 10 of FIG. 1 includes a plurality of ceiling-mounted light sources. More generally, all the light sources could be ceiling-mounted, and/or wall-mounted. More generally, the light sources may be supported in lamp holder fixtures, or resting or the floor or on furniture, in coves, suspended from supports, or so forth. The at least one light source 10 preferably includes a plurality of light sources distributed over wall(s) and/or the ceiling so as to apply the light to most or all of the ambient air and surfaces in the environment 2. Complete coverage may not be necessary, however, if the ambient air in the environment 2 is circulating so that air in any “dead” areas that are not irradiated by the light will move by convection or other circulation into irradiated areas.

The light emitted by the at least one light source 10 may be UVC light, UVA light, violet light, or some combination thereof. Depending on the type of light source 10, the light may be narrow-band light, e.g. predominantly a single discrete emission line or a set of discrete emission lines or may be broad-band light. Preferably the intensity of the light emitted by the at least one light source 10 is effective to achieve at least 90% inactivation of (at least) a pathogen of interest in the ambient air within about eight hours, or more preferably within about two hours, or more preferably in less than one hour.

With reference to FIG. 2, in some embodiments each light source 10 comprises one or more light emitting diodes (LEDs) 20, for example disposed on a printed-circuit board or other substrate 22 and optionally mounted in a housing (not shown). The LEDs 20 may all be of the same type (e.g. all LEDs emitting light having a peak in the UVA) or may be a mixture of LEDs of different types (e.g., some LEDs emitting light having a peak in the UVA and some LEDs emitting light having a peak in the UVC). In some embodiments, there may be as few as a single LED 20 disposed on the substrate 22. The substrate 22 may optionally be coated with a diffuse or specular reflective layer such as an aluminum layer, a silver layer, a foam Teflon (e.g. ePTFE from W.L. Gore) layer, a thin-film optical coating, or so forth in order to increase the light emission efficiency.

With reference to FIG. 3, in some embodiments each light source 10 comprises a gas discharge lamp 30, optionally further including a collecting reflector 32 with a reflecting surface such as an aluminum surface, a silver surface, a foam Teflon (e.g. ePTFE) surface, a thin-film optical coating, or so forth in order to increase the light emission efficiency. For example, the lamp 30 may be a mercury (Hg) lamp, such as a medium-pressure Hg lamp, or a low-pressure Hg lamp, which emits light in the UVC range.

The light source 10 may optionally include additional features, such as a lightbulb socket 34 for mechanically and electrically connecting the light source 30 to A.C. electrical power, or a spectral filter 36 to selectively emit only a desired spectral line or lines of a multiple-peak gas discharge lamp emission spectrum. If the UV intensity output by the illustrative gas discharge lamp 30 is too high to ensure safety of the occupants, the spectral filter 36 may additionally or alternatively integrate or be deployed in combination with a neutral density filter or the like to reduce the UV radiation intensity. While the illustrative lightbulb socket 34 is an Edison screw lightbulb socket 34, another type of lightbulb socket may be used (e.g., bayonet socket, bi-post socket, bi-pin socket) or some other type of electrical connector or connection may be employed (e.g., a pigtail for wiring to an electrical power source), or the light source 10 may include an on-board battery. It will be appreciated that the light source 10 may also include suitable electrical power conditioning circuitry, e.g. an electrical ballast circuit for driving the Hg lamp 30, or LED driver circuitry disposed on or embedded in the substrate 22 in the case of an LED-based light source such as that of FIG. 2.

With continuing reference to FIGS. 2 and 3, in some embodiments one or more occupancy sensors 40, 42 is/are provided, which is/are configured to detect occupancy of the environment 2; and electronics typically including an electronic processor (not shown, e.g. a microprocessor or microcontroller and ancillary electronics such as a RAM and/or other memory chip, discrete circuit elements, and/or so forth) that is configured (e.g. programmed by software or firmware stored in a memory chip and executable by the microprocessor) to control the at least one light source 10 to output the disinfecting light into the environment 2 based on the occupancy of the environment 2 detected by the sensor 40, 42. By way of non-limiting illustration, the LED-based light source of FIG. 2 includes a motion sensor, thermopile, ultrasonic sensor, or other occupancy sensor(s) 40 for detecting occupancy of the environment 2 by detecting motion in the environment. The motion sensor 40 may comprise any suitable motion sensor, for example a passive infrared (PIR) motion sensor, a microwave motion sensor, an ultrasonic motion sensor, a camera-based motion sensor, and/or so forth. A camera-based, or imaging, sensor may determine the density or proximity of occupants and respond with higher or lower UV doses as appropriate. As a further non-limiting illustration, the sensor may comprise a microphone 42 as shown in FIG. 3, which detects occupancy based on detected vocalization. As a further non-limiting illustration, the sensor may comprise a particle detector, which detects the density of small sized particles and adjusts the light intensity responsive to the expected density of virus and/or bacterial particles in the air.

The illustrative sensor 40, 42 is integrated into a light source 10; if the electronic processor is also integrated into the light source 10 then this can provide a single unitary device that both emits the UV light for disinfection and detects occupancy and controls that UV light based on the occupancy. In other embodiments (not shown), the sensor may be a separate component from the light source(s) 10, and the electronic processor may be integral with the light source(s) 10, or may be integral with the sensor component, or the electronic processor may be a third component separate from both the light source(s) and the sensor component. Such “distributed” implementations may advantageously allow the electronic processor to receive sensor signals from a number of sensors distributed in the environment 2 so as to more accurately assess occupancy of the environment 2.

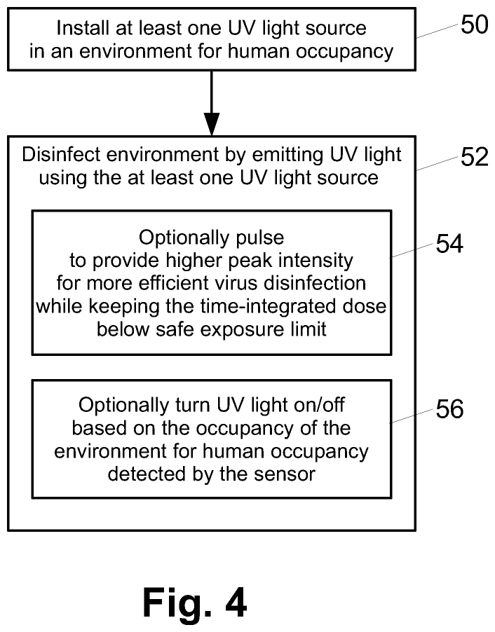

With reference now to FIG. 4, a disinfection method suitably performed using the light source(s) 10 is described. In an operation 50, the light source(s) are installed in the environment 2 for human occupancy. This entails physically mounting the light sources, and electrically connecting the light sources to electrical power (e.g., connecting the lightbulb socket 34 to a pre-existing lighting receptacle, installing a battery if the light source is battery powered, or wiring a pigtail to electrical power, or so forth). In the installation operation 50, care should be taken to provide sufficient coverage of the volume of ambient air in the environment 2, so that most or all of this volume is irradiated by the disinfecting light emitted by the light source(s) 10. Additionally, care should be taken to ensure that persons in the environment 2 are not exposed to excessive ultraviolet light by being too close to the light source(s) 10. For example, the light source(s) 10 can be designed for ceiling mounting, and the light source(s) 10 can be designed so that when thusly spaced from the one or more surfaces 12 by (about) the ceiling height, this distance is large enough for the light to have irradiance at the one or more surfaces 12 below the exposure threshold (e.g., 30 J/m² or less of actinic-weighted irradiance, or 60 J/m² or less over an eight hour period in some embodiments, as further explained elsewhere herein).

For example, in one approach the light sources 10 have preset controls that are set by the manufacturer at the factory or during installation. In an embodiment with no microprocessor, the preset control of each light source 10 may include only an intensity setting, for example diagrammatically implemented as an intensity setting dial 13 shown in FIG. 2. While the dial 13 is illustrated on the side of the substrate 22 of the light source 10, in some embodiments it may be more conveniently placed on a backside of the substrate 22, as it will only be set at the factory or during installation, or at infrequent maintenance intervals. Rather than a dial, the intensity setting input 13 may be a knob, recessed hex head operating a rheostat, or so forth. The manufacturer sets the intensity at the factory or during installation based on the geometry of the environment 2 and the spacing of the light sources 10 (e.g., spacing across the ceiling 4, see FIG. 1). In particular, for ceiling mounted light sources 10 as in FIG. 1, persons occupying the environment 2 will receive highest intensity at “head level”—that is, the person when standing normally on the floor 6 will receive the highest intensity at the head and eyes which are closest to the ceiling 4. While persons vary in height, typically “head level” for tall persons is about 2 meters above the floor 6. As a non-limiting illustrative example, to provide a safety margin, in some contemplated designs a design-basis “head level” of 2.1 meters may be defined. Hence, if a single light source 10 is considered then the preset intensity control is suitably set for a light source-to-head level distance of H−2.1 meters where H is the ceiling height, i.e. the shortest distance between the ceiling 4 and floor 6. This assumes the light source 10 is mounted flush with the ceiling 4—if the light sources 10 have some drop-down mounting where the light source level is a drop-down distance d below the ceiling 4 then the light source-to-head level distance of H−d−2.1 meters. In some embodiments, it is contemplated for the light source for disinfection to have no other control besides the intensity setting input 13.

The maximum permissible intensity for safe occupation can be determined as follows. The light source 10 outputs light with a known peak wavelength or spectrum. An actinic action spectrum is also known, e.g. the published IESNA Germicidal action spectrum may be used. The dose time period over which the actinic dose exposure limit is to be calculated is also chosen. This is typically taken to be 8 hours assuming an 8-hour work shift, but for example in the case of a storage room that is infrequently accessed a shorter dose time period may be appropriate. An actinic dose limit is also known a priori, typically provided by jurisdictional safety regulations. For example, presently (circa year 2020) in the United States, the actinic dose limit is defined as follows: the effective integrated spectral irradiance Eₛ should not exceed 30 J/m², where Eₛ is defined as the quantity obtained by weighting spectrally the dose (radiant exposure) over the dose time period according to the actinic action spectrum value at the corresponding wavelength. Since the dose is the time-integrated irradiance, the actinic dose limit divided by the dose time period yields the maximum permissible irradiance for safe occupation at any location in the irradiated space.

Finally, given the spatial irradiance distribution output by the light source 10 (e.g., a Lambertian spatial distribution in one non-limiting illustrative example), the fractional irradiance decrease as a function of light source-to-head level distance can be computed and tabulated or presented as a plot (where for example, the irradiance at light source-to-head level distance=0 can be normalized to one, and the fractional irradiance monotonically decreases with increasing light source-to-head level distance). Then given the installation-specific light source-to-head level distance and the computed maximum permissible irradiance for safe occupation, the light source irradiance at light source-to-head level distance=0 can be preset using an intensity control knob (e.g. a rheostat controlling electrical current of the LEDs 20 of the light source of FIG. 2) or can be programmed into nonvolatile memory of an electronic controller (e.g. microprocessor-based controller) of the light source 10 that controls the electrical current of the LEDs 20 so as to ensure that the irradiance at the light source-to-head level distance is less than the maximum permissible irradiance for safe occupation. Preferably, this adjustment is done using a photosensor to measure/confirm the adjusted irradiance.

If there are multiple light sources 10 as in the example of FIG. 1, and if the light from neighboring light sources spatially overlap, then a further scaling factor is applied to correct for the spatial overlap. For example, if it is calculated for the geometry (including the spacing of the light sources 10 over the ceiling 4, the Lambertian or other light distribution of the light sources, and the light source-to-head level distance) that the irradiance at maximum overlap is twice that of a single light source 10, then the above computed irradiance should be reduced by 50% to ensure that the irradiance at the light source-to-head level distance remains less than the maximum permissible irradiance for safe occupation at the point of maximum light overlap.

To ensure that the end-user does not change the preset intensity, or misuse the light source by (for example) installing it at a smaller light source-to-head level distance than the intensity was set for, in some embodiments the preset is designed to not be user-adjustable (e.g., if set in nonvolatile memory firmware, a password or other electronic security may be employed to ensure adjustment only by an authorized person), or at least not easily user-adjustable (e.g., a rheostat may be adjustable using a special hex key or the like); and/or the light source 10 after adjustment may be prominently labeled with a warning label indicating the minimum light source-to-head level distance for which the light source is designed. (There is no safety hazard with using the light source at a larger light source-to-head level distance than the intensity was set for, hence a minimum light source-to-head level distance is suitably specified on the warning label).

With continuing reference to FIG. 4, in an operation 52 the ambient air, surfaces and materials of the environment 2 are disinfected by emitting UV light using the at least one UV light source 10. As will be described in greater detail elsewhere herein, the light source(s) 10 are designed to provide sufficient irradiance to provide effective viral disinfection while ensuring the UV light exposure remains below the safety dose threshold for a typical 8-hour workday. As further indicated in FIG. 4, in some embodiments this balancing of viral disinfection efficacy versus ensuring occupant safety is achieved in part by pulsing the UV light to provide higher peak intensity for more efficient virus disinfection while keeping the time-integrated dose below the safe exposure limit. Such pulsing can be performed by the electronic controller, or can be implemented by an analog circuit that applies electrical pulses to the LEDs 20 or Hg lamp 30. In some non-limiting illustrative embodiments, the light source(s) 10 are configured to generate the light as pulses having pulse width of 1 second or less and pulse spacing of at least 10 seconds. This reflects the fact that the inactivation of many pathogens is not reciprocal, i.e., a measured dose [J/m²] delivered in a short time may be more effective than the same dose delivered over a longer time; whereas, the safety hazard is a function of the time-integrated exposure dose. For (as just one example) 1 second pulses spaced apart by 10 seconds, the duty cycle is only 10% leading to an order-of-magnitude reduced time-integrated dose. Alternatively (as just one example), 1 second pulses can be made at 10 times higher irradiance to achieve better viral disinfection while maintaining the same time-integrated dose as a continuous irradiance at the time-averaged level.

With continuing reference to FIG. 4, optionally the sensor 40, 42 is used to turn the UV light on or off based on the occupancy of the environment 2. If the dominant viral transmission vector is by way of respiratory droplets, and the bare virus particles after droplet evaporation may stay suspended for about two hours on average (or possibly for about 1 hour, or about 4 hours, or about 8 hours), then the occupancy-based control may be designed to turn the UV light on, or increase the intensity of the UV light, in response to detected occupancy, and then turn it off (or reduce the intensity) two hours after the detection of a cessation of occupancy. This can reduce energy consumption—however, energy consumption may be negligible due to the low intensity of the UV light emitted by the light source(s) 10. A more significant advantage of this occupancy-based control is to reduce the UV dose to surfaces inside the environment 2. For example, some fabrics, furniture covers, plastics, and the like can become discolored over time due to UV exposure. In the case of a space that is only occupied during an 8-hour work day, and possibly only for some small portion(s) of that work day (for example, a conference room that is only used for a couple hours during the work day), this approach of occupancy-based control can greatly reduce the UV exposure of surfaces, thereby reducing UV-induced surface discoloration.

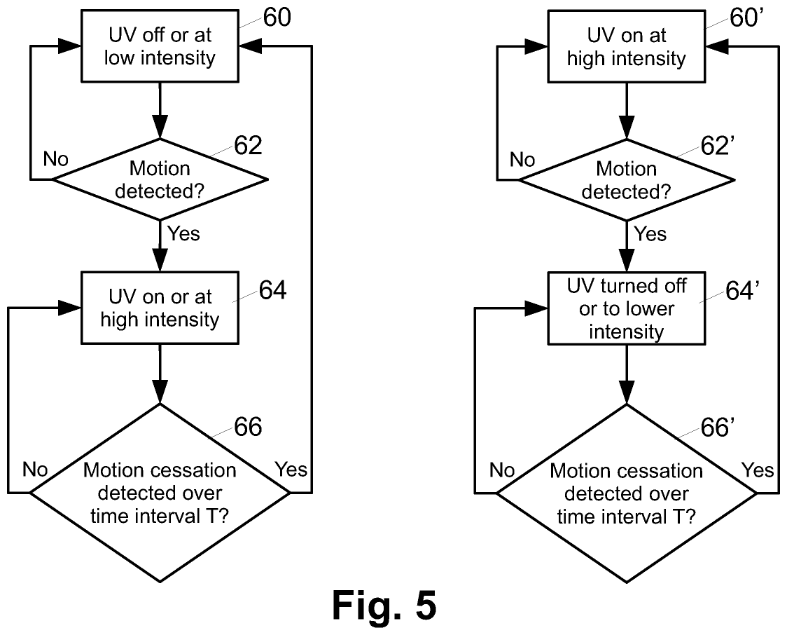

With reference to FIG. 5, two illustrative examples of occupancy-based control using the motion sensor 40 of FIG. 2 are described. With reference first to the left-hand flowchart, at a state 60, the light source(s) 10 are assumed to be off or operating at low intensity. At a decision 62, the motion sensor 40 is monitored, and as long as motion is not detected the light source(s) 10 are kept in the state 60. When at the decision 62 motion is detected, then the light source(s) 10 are switched to a state 64 in which the light source(s) 10 are on or brought up to emit the UV light at a higher intensity. Thereafter, at a decision 66, the motion sensor 40 is again monitored to detect when motion ceases for a time interval T. As long as this condition is not met, the light source(s) 10 are kept in the state 64 to provide viral disinfection (or increased viral disinfection). When at the decision 66 it is determined that motion has ceased for the time interval T, then the light source(s) 10 are switched back to the state 60 in which the light source(s) 10 are off or reduced to the low intensity. The time interval T is suitably chosen based on the (statistical) settling time of virus particles from the ambient air onto the floor and other surfaces. For coronavirus particles, this settling time has been estimated to be about 2 hours; hence, the predetermined time T may suitably be between one and three hours inclusive in some embodiments. The time interval may be chosen for a specific implementation based on the statistical settling time of the virus particles to be disinfected balanced by factors such as the desire to reduce UV damage to surfaces in the environment 2. In some embodiments, it is contemplated for the time interval T to be set to zero, in which case the light source(s) 10 are switched back to the state 60 in which the light source(s) 10 are off or reduced to the low intensity immediately upon detection of the cessation of motion at the operation 66.

With continuing reference to FIG. 5 but now referencing the right-hand flowchart, the control may also reduce or turn off the UV intensity in response to detected motion. By this alternative approach, the disinfection system may apply UV at an intensity such that the light emitted by the light source(s) 10 is effective to produce an actinic dose at a target plane in the environment above the 30 J/m² threshold over an eight hour period, but to do so only when the environment 2 is unoccupied. To this end, at a state 60′, the light source(s) 10 are assumed to be on and operating at high intensity (again, optionally at an intensity such that the light emitted by the light source(s) 10 is effective to produce an actinic dose at a target plane in the environment above the 30 J/m² threshold over an eight hour period). At a decision 62′, the motion sensor 40 is monitored, and as long as motion is not detected the light source(s) 10 are kept in the state 60′. When at the decision 62′ motion is detected, then the light source(s) 10 are switched to a state 64′ in which the light source(s) 10 are turned off or reduced to a lower intensity, e.g. to an intensity such that the light emitted by the light source(s) 10 is effective to produce an actinic dose at a target plane in the environment that is below the 30 J/m² threshold over an eight-hour period. Thereafter, at a decision 66′, the motion sensor 40 is again monitored to detect when motion ceases for a time interval T. As long as this condition is not met, the light source(s) 10 are kept in the state 64′ to provide safety for the persons occupying the environment 2. When at the decision 66′ it is determined that motion has ceased for the time interval T, then the light source(s) 10 are switched back to the state 60′ in which the light source(s) 10 are on and emitting at the high intensity. Here, the time interval T may be set to zero, or may be set to a value chosen to allow for some error in the occupancy sensing operation 66′. For example, a time interval T of two minutes may be chosen to ensure that the light source(s) 10 are not switched to the state 60′ due to a period of inactivity by the occupants.

With continuing reference to FIG. 5 and further referring back to FIG. 1, in another contemplated embodiment the light sources 10 include microprocessors as described, and further include short-range wireless transceivers (e.g. Bluetooth™ or ZigBee radios) whereby the light sources 10 are configured as a wireless mesh network 68 (as diagrammatically indicated in FIG. 1). Alternatively, the light sources 10 include microprocessors as described, and are further physically wired together to form a wired mesh network. Other wireless or wired network topologies are also contemplated. Optionally, a network controller 69 may be provided. An illustrative network controller 69 is diagrammatically shown in FIG. 1 as a ceiling-mounted unit, e.g. a computer or other microprocessor-equipped electronic data processing device that may be located in the environment 2 as shown. In some embodiments, the network controller 69 may be a designated one of the light sources 10 which is equipped with (typically) greater electronic data processing capacity than the other light sources 10 and hence serves as a central hub of the light source communication network. In further embodiments, the network controller 69 may be or include a keyboard, mouse, touch-sensitive display, or other user input device(s), and a display. For example, the network controller 69 may be or include a desktop computer running a lighting software control package. In yet further variant embodiments, the network controller 69 may be partly or wholly remote, e.g. a server computer connected with the light source communication network 68 via WiFi or wired Ethernet or the like.

The resulting wireless or wired light source communication network 68 (e.g., light source mesh network) enables the light sources 10 to share data acquired by their respective occupancy sensors 40, 42. This advantageously allows for aggregating the occupancy sensor data to make more accurate determinations as to occupancy of the environment 2. For example, the higher intensity that would result in a dose above the actinic dose exposure limit for the dose time period may be applied only if every occupancy sensor of every light source agrees that the environment 2 is unoccupied. In another embodiment, the higher intensity may be applied only if a majority (i.e. greater than 50%) or defined supermajority (i.e., greater than X % where X % is a design parameter higher than 50%) of the occupancy sensors agree that the environment 2 is unoccupied.

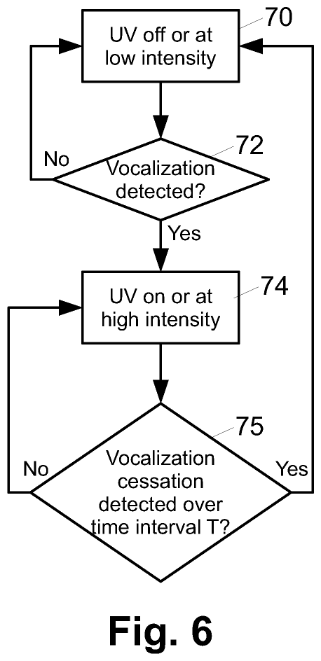

With reference to FIG. 6, an illustrative example of occupancy-based control using the microphone 42 of FIG. 3 is described. At a state 70, the light source(s) 10 are assumed to be off or operating at low intensity. At a decision 72, the microphone 42 is monitored, and as long as vocalization is not detected the light source(s) 10 are kept in the state 70. In a simple embodiment, any detected sound whose amplitude is above some minimum threshold is taken to be a detection of vocalization. In a more complex embodiment, spectral filtering, sound duration, or other automated analysis of the detected sound may also be applied so as to reduce likelihood that spurious noise caused by the HVAC system or other noise sources is misinterpreted as vocalization. When at the decision 72 vocalization is detected, then the light source(s) 10 are switched to a state 74 in which the light source(s) 10 are on or brought up to emit the UV light at a higher intensity. Thereafter, at a decision 75, the microphone 42 is again monitored to detect when vocalization ceases for a time interval T. As long as this condition is not met, the light source(s) 10 are kept in the state 74 to provide viral disinfection (or increased viral disinfection). When at the decision 75 it is determined that motion has ceased for the time interval T, then the light source(s) 10 are switched back to the state 70 in which the light source(s) 10 are off or reduced to the low intensity. The time interval T is suitably chosen as described for the motion sensor-based control of FIG. 5. An advantage of using vocalization detection for the control is that respiratory droplet mediated transmission is most likely in response to an infected person talking, singing, coughing, sneezing, or engaging in some other vocalization. On the other hand, if an infected person merely passes through the environment 2 without vocalizing, the likelihood of transmission is much lower compared with the case of vocalization. Hence, the vocalization-based control may provide more well-tailored application of the UV disinfection for these viruses. In some variant embodiments (not shown), the control approach of FIG. 6 may be adjusted to, for example, deliver a short period (e.g. 5-20 minutes in some embodiments) of higher intensity UV light in response to a detected loud vocalization such as a cough, singing, shouting, or the like which (if done by a virus-infected person) is likely to expel a higher concentration of virus particles into the ambient air as compared with soft speaking. In another embodiment, the motion, occupancy, or microphone sensors may be spatially resolved thereby directing only those UV light sources that are most directly irradiating the source of the motion, occupancy or sound to be irradiated, or to receive enhanced irradiation.

It will be appreciated that a variant of the embodiment of FIG. 6 analogous to that of the right-hand flowchart of FIG. 5 may be employed, in which the UV is on at high intensity and is turned off or to lower intensity in response to detection of occupancy of the environment 2.

In general, using a microphone 42 as an occupancy sensor provides for performing actions determined based on the type of sound detected. For example, detecting a sound indicating human presence may cause the light source 10 to adopt its occupied (i.e., not vacant) state and lower its long-term irradiance levels accordingly. On the other hand, detection of a specific sound which indicates a possible aerosol emission event (e.g., a cough, shout, et cetera) produced by an occupant of the environment 2 may cause a brief increase in intensity to increase pathogen kill within the aerosol before it infects another occupant of the environment 2. This increase in intensity amounts to “spending” a portion of the actinic dose budget for the dose time period (e.g., 8 hours in some embodiments) determined for the occupied state; the excess dose delivered during this brief time period should then be subtracted from the remaining actinic dose budget for the dose time period. For example, if the brief increase in intensity over a brief time period after an aerosol emission amounts to 5% of the actinic budget, then the actinic dose for the remaining portion of the dose time period is suitably lowered accordingly.

Depending upon the nature of the environment 2 for human occupancy, the occupancy sensor(s) 40, 42 can take other forms. For example, in an intensive care unit (ICU), bedroom, or other setting in which occupants may be present but sleeping or unconscious in a bed, typical occupancy sensors based on detecting movement or the like may be ineffective or of reduced effectiveness. In such situations, the occupancy sensor(s) 40, 42 may, for example, include thermal imaging sensors that can detect a motionless (e.g. sleeping) person by detecting their body heat. In another approach, the occupancy sensor(s) 40, 42 may include weight sensors incorporated into beds of the ICU or bedroom, which detect the weight of a human bed occupant. This latter example would be one where the sensor is not incorporated into the light source, but rather the weight sensor would be incorporated into beds disposed in the environment 2. These bed weight sensors could be wired to the light source(s) 10, or if the light source communication network 68 is provided then the bed weight sensors could communicate bed occupancy measurements to the light sources 10 via the communication network 68.

As yet another example of a different type of occupancy sensor, in a secure building setting the occupancy sensor(s) 40, 42 could include a master key switch, electronic lock, or other electronic entry security device implemented at a door or other accessway to the environment 2 for human occupancy. For example, in an office setting, a security guard may perform a walk-through inspection at the end of the workday and may then activate the electronic lock to secure the office at the end of the inspection. In such a case, the electronic lock can serve as an occupancy sensor, since when the lock is disengaged this at least permits occupancy, while when the lock is engaged the office space should be unoccupied. A similar example is a people counting system that is employed in some secure settings, that may for example count passages of persons into/out of the environment 2. (For example, two laser/photodiode sensors can be placed at the inside and outside positions of the door, with each laser/photodiode sensor detecting a person crossing the laser beam by detecting the breaking of the beam. A triggering of the outside laser/photodiode sensor followed by a triggering of the inside laser/photodiode sensor indicates a person entering the environment 2; whereas, a triggering of the inside laser/photodiode sensor followed by a triggering of the outside laser/photodiode sensor indicates a person leaving the environment 2. These enter/exit events can be counted to determine occupancy of the environment 2). As still yet another example, if occupants of the environment 2 are required to wear RFID badges that are monitored by RFID readers, then this system can be used to determine occupancy of the environment 2.

Again, the foregoing are merely examples. It will be appreciated that various of these different types of occupancy sensors may be used in various combinations. Furthermore, the reliability of the different types of occupancy sensors may vary. For example, an RFID tag-based occupancy sensor may fail if a person fails to wear an RFID badge. Laser/photodiode sensor based counting systems can fail if for example two people enter or exit closely behind one another. As already noted, motion-based occupancy sensors can fail if the occupant is motionless (e.g., sleeping). Again, the occupancy sensor data can optionally be aggregated (e.g., by communication via the light source communication network 68) to make more accurate determinations as to occupancy of the environment 2, e.g. using a voting system requiring a majority or supermajority of the sensors to agree on non-occupancy before the light source(s) 10 are permitted to exceed the actinic dose limit.

The light source communication network 68, if provided, may be utilized to implement other capabilities. For example, as previously described in some embodiments the light sources 10 are preconfigured for a specific light source-to-head level distance and a specific dose time period. Since for a given irradiance the dose scales with the dose time period, if the dose time period is (for example) doubled then the irradiance should be halved to ensure that the actinic dose exposure limit is not exceeded over the doubled dose time period. This scaling could be implemented via the light source communication network 68 and the network controller 69. For example, a lighting control software package running on the network controller 69 may enable the user to increase the dose time period by a factor of X, and the lighting control software then automatically adjusts the intensities of the light sources 10 by a factor of 1/X. In this way, scheduled employee overtime can be accommodated in a straightforward fashion. In such embodiments, the lighting control software package preferably has access security controlled by a password, two-factor authentication (2FA), or the like to ensure that only authorized personnel are permitted to adjust the dose time period.

The network controller 69 may also collect occupancy data from the occupancy sensors 40, 42 and provide occupancy reports via a display (not shown) of the controller 69. For example, this may plot occupancy information as a function of time of day, day of week, or other time intervals/groupings. A human supervisor can then use this information to re-configure the light sources 10 over the network 68.

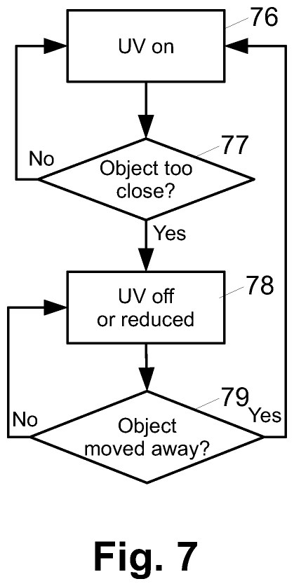

With reference to FIG. 7 and reference back to FIGS. 2 and 3, in some embodiments, the sensor(s) 40, 42 built into the light source 10 include a proximity or distance sensor such as a LIDAR, ultrasonic proximity sensor, thermal proximity sensor, or the like. In such an embodiment (which is assumed to further include a microprocessor), if the light source 10 is presently operating to emit UV light into the environment (operation 76) and in an operation 77 the proximity or distance sensor detects any object closer than the configured light source-to-head level distance (or other programmed distance), then in an operation 78 any light presenting an actinic hazard that is emitting from the light source 10 is automatically turned off. The UV emission remains off until in an operation 79 it is detected that the object has moved away to at least the configured light source-to-head level distance, at which point process flow returns to operation 76 to turn the UV emission back on. This provides a failsafe interlock in case (for example) ladder-borne maintenance personnel come close to the light source. The shutoff condition can also take other forms, e.g. shutoff of the UV emission if an object is detected that is closer than some fixed distance from the light source 10 (regardless of the configured light source-to-head level distance). If the light source 10 is configured to emit UV at above the actinic dose limit when the occupancy sensor(s) 40, 42 indicate the environment 2 is unoccupied, then the proximity or distance sensor can also serve as a back-up occupancy sensor by reducing the UV irradiance to at or below the actinic dose limit if the proximity or distance sensor detects an object in proximity to the light source. Note that in the case of a proximity sensor that does not actually measure distance, the proximity sensor effectively detects an object closer than a fixed distance which is the distance at which the proximity sensor is triggered. Because the eyes tend to be more sensitive to ultraviolet radiation than the skin, in some variant embodiments the proximity sensor include gaze sensors that detect a gaze toward the light source and trigger off such detection (alone or in combination with proximity detection).

With continuing reference to FIG. 7, in a variant embodiment the operation 77 uses the proximity or distance sensor to measure the distance of the object from the light source 10, and as the object comes closer than the configured light source-to-head level distance (or other programmed distance), then the operation 78 dims the light source 10 based on how close the object is, rather than turning it off. This approach may be useful if, for example, the light source 10 is being used in a setting where the objects will usually be further away than the configured light source-to-head level distance but may occasionally come closer. An example of such an environment might be a preschool or grade school classroom where the configured light source-to-head level distance might be configured for small children as most occupants are small children, but an adult or unusually tall child may also be present in the classroom. In this case, if the adult (e.g. teacher) walks underneath the light source 10 then the proximity sensor will detect this and reduce (but not necessarily turn off) the light source 10. In this case, a graded approach may be used—if the object distance measured at operation 77 corresponds to an adult passing or standing underneath the light source 10 then it may be dimmed, but if the object comes still closer (for example, due to a school janitor working on a ladder near the light source) then it may be turned off.

Whereas the above describe control systems and methods provide for controlled irradiance of an environment, it may be preferable to control the irradiance for an individual, rather than for an occupied space. For example, an individual may wear a UV dosimeter or may possess a smart phone or other device in communication with the UV light sources, so that the light sources may respond with higher or lower intensities responsive to the cumulative dose of that individual over a 24 hour period (or an 8-hour period or other design-basis dose time period for which the actinic dose limit is defined). If multiple individuals occupy the same UV-irradiated space, then the light sources may respond with light intensity geared to that individual most likely to reach the actinic limit in the 8-hour period. One example of the use of such a system and method may be in the context of an individual who has worked alone in a space for several hours, and may have elected to lower the UV intensity or to turn it off during that period, who then attends a meeting with other people and chooses to have the UV light source(s) in closest proximity to the individual emit a higher intensity during the meeting. In another example, all workers in an office, warehouse, or other workspace wear dosimeters with wireless connectivity to the light source communication network 68. For example, each dosimeter may be mounted on a headband worn by a worker, so as to record UV exposure to the worker's face (thereby recording a close approximation of the UV exposure to the worker's eyes). In one such embodiment, the dosimeters record whether a maximum UV dose has been received at the dosimeter. In this example, the dosimeters include circuitry to send an alert via the network 68 if the actinic dose limit (or other chosen UV exposure limit) is exceeded, and the network controller 69 shuts off the UV sources in the room where that worker is located in response to the alert. In a more elaborate embodiment, the dosimeters include circuitry (e.g. a programmed microprocessor) to send actual dose readings to the network 68 as samples acquired (e.g.) every few seconds, and the network controller 69 adjusts the intensities of the UV light sources in a given room based on the highest dose thus far received by any worker in that room.

In another example, if the occupants of the environment 2 are required to wear RFID badges that are monitored by RFID readers and that provide unique identification of individual occupants (or, equivalently, some other type of identification badges is employed, e.g. infrared badges read by infrared readers), then this occupant identification system can be used to determine the time-integrated exposure (i.e. the dose received up to present time) of individual occupants. Here the time-integrated exposure is calculated based on the UV light emitted in the room or rooms the individual occupies over time. The UV irradiance received by the individual is time-integrated to obtain the dose up to the present time. If this dose reaches the actinic dose limit, then from that point on any room in which that individual is present has its UV light turned off. In a more elaborate embodiment, the network controller 69 adjusts the intensities of the UV light sources in the room based on the highest dose thus far received by any worker in that room. Even more generally, the intensity of the ultraviolet light emitted by the one or more light sources 10 is controlled based on ultraviolet doses received by occupants determined from the data indicative of occupancy of the environment including ultraviolet doses received by the occupants computed based on tracking of the occupants using the identification badges.

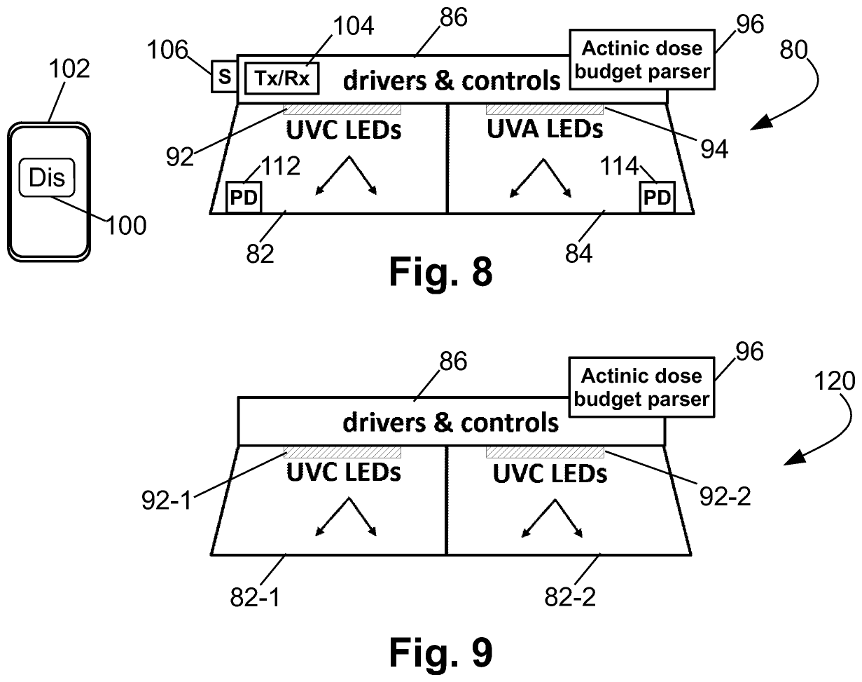

With reference now to FIG. 8, an illustrative example is diagrammatically shown of a multispectral (here UVC and UVA) light source for disinfection which is implemented as a single light fixture 80 that includes UVC LEDs 82 and UVA LEDs 84 along with driver and control electronics 86. In one suitable physical layout, the UVC LEDs 82 may be disposed on a first (UVC) printed circuit board (PCB) 92 which optionally may include power conditioning circuitry; and the UVA LEDs 84 may be disposed similarly disposed on a second (UVA) PCB 94 which again optionally may include power conditioning circuitry. Alternatively, the UVC and UVA LEDs may be disposed on a single PCB, or the UVC (or UVA) LEDs may be distributed across multiple PCBs.

The driver and control electronics 86 may optionally include an electronic processor (e.g. a microprocessor or microcontroller) programmed to implement an actinic dose budget parser 96 that controls the outputs of the UVC LEDs 82 and the UVA LEDs 84 based on a control input. For example, the driver and control electronics 86 may be configured to implement the actinic dose budget parser 96 to control the intensity and/or spectrum of the ultraviolet light emitted by the one or more light sources based on the data indicative of the environment acquired by the one or more sensors 40, 42 to control an irradiance of the one or more light sources at a defined location in the environment 2 for human occupancy. In an example, if the light source(s) 10 are ceiling-mounted as shown in FIG. 1 and the environment 2 is a room, then the defined location in the environment 2 may be a “head level”, such as a plane that is 2.1 meters above the floor 6. Some other defined location may be used depending on the environment 2—as another example, if the environment 2 is an office with a desk then the defined location may be the location of a person sitting at the desk. In another embodiment, the actinic dose budget parser 96 is implemented by analog circuitry or by digital circuitry that does not include an electronic processor. In general, the actinic dose fraction delivered by each UV LED set 82, 84 is controlled by adjusting the electrical current (or voltage) applied to the LEDs to adjust the output intensity. In some embodiments, the control input is a manually supplied control input, e.g., provided wirelessly via a control application 100 running on a cellular telephone or other mobile device 102 operated by a building manager or other authorized person which transmits the control signal that is wirelessly received by a wireless transceiver (or wireless receiver) 104 of the driver and control electronics 86. Alternatively, the manually supplied control input may be implemented as a manual switch or other manual control built into the fixture 80. For example, the actinic dose budget control may in some embodiments have only two settings: (1) one setting to relatively increase the UVC actinic dose fraction over the UVA actinic dose fraction to emphasize virus inactivation over bacteria inactivation; and (2) the other setting to relatively increase the UVA actinic dose fraction over the UVC actinic dose fraction to emphasize bacteria inactivation over virus inactivation. In this case, the manual control could be a two-setting switch that can be set to: Setting 1—virus inactivation; or Setting 2—bacterial inactivation. (It should be noted that in this embodiment the virus inactivation setting may optionally still have some non-zero UVA actinic dose fraction to provide some bacterial inactivation; and likewise the bacteria inactivation setting may optionally still have some non-zero UVC actinic dose fraction to provide some viral inactivation). Other embodiments are contemplated, e.g. a three-position switch, a toggle switch, et cetera.

In yet another contemplated embodiment, the control input is automatically provided by one or more biosensors 106 that are integrated with the fixture 80 (as shown) or separate from the fixture but in wired or wireless communication with the electronics 86. The biosensor(s) 106 may employ any conventional biosensing technology (e.g., electrochemical, ion channel switch, fluorescent biosensor, et cetera) to detect a specific pathogen or class of pathogens. The biosensor(s) may be mounted on the fixture 80 as shown or may be mounted elsewhere and connected to the fixture electronics 86 by a wired (e.g. USB cable or DALI) or wireless (e.g. WiFi, Bluetooth, or Zigbee) connection.

To provide feedback control of the intensities of the UVC LEDs 82 and UVA LEDs 84, respectively, it is optionally contemplated to incorporate a UVC-sensitive sensor, e.g. a photodiode 112 to directly measure the UVC irradiance and likewise a UVA-sensitive sensor, e.g. a photodiode 114 to directly measure the UVA irradiance. In another non-limiting illustrative approach, open-loop control can be used based on a UVC (or UVA) output intensity versus drive current (or voltage) calibration that is predetermined for the specific fixture 80 or for that make/model of fixture 80.

With reference to FIGS. 9-13, some additional embodiments of multispectral light sources for performing disinfection in an occupied (or, in some embodiments, unoccupied) space are described. FIG. 9 illustrates a single fixture 120 that provides UVC disinfection light at two different wavelengths by way of a first set of UVC LEDs 82-1 emitting at a first UVC wavelength λ₁ that may be disposed on a first PCB 92-1 which optionally may include power conditioning circuitry; and a second set of UVC LEDs 82-2 emitting at a second UVC wavelength λ₂ (where λ₁≠λ₂) that may be disposed similarly disposed on a second PCB 92-2 which again optionally may include power conditioning circuitry. Alternatively, the two sets of UVC LEDs 82-1, 82-2 may be disposed on a single PCB. In this embodiment, the two UVC wavelengths λ₁ and λ₂ are selected to provide effective inactivation of a target pathogen or class of pathogens. In one non-limiting illustrative example, λ₁=255 nm and λ₂=280 nm. Optionally, the actinic dose budget parser 96 is included with the driver and control electronics 86 to control the relative actinic dose fractions of the respective UVC LEDs 82-1, 82-2 based on a control input such as already described with reference to FIG. 8.

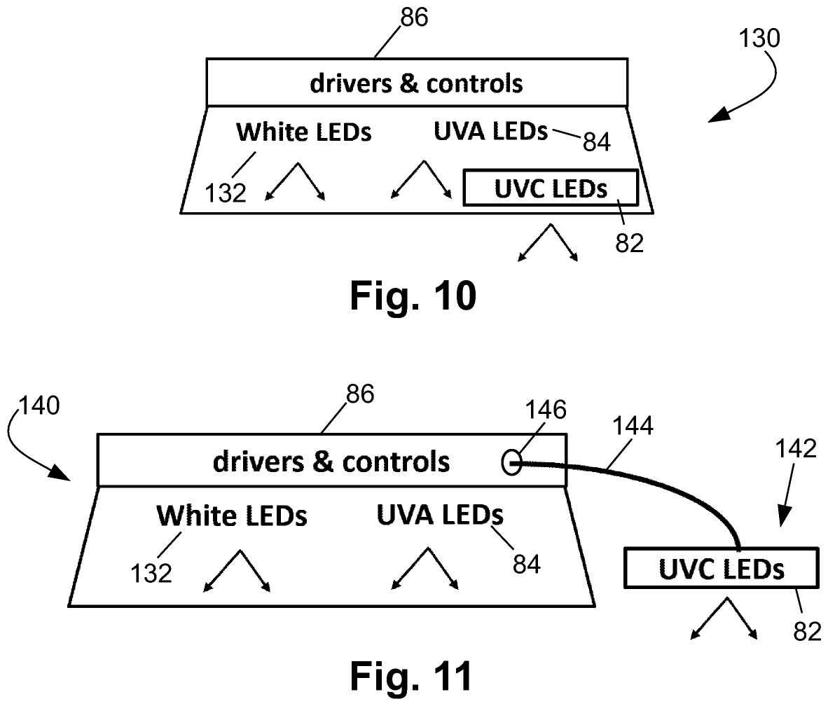

FIG. 10 illustrates another example, in which a single fixture 130 provides both disinfection by way of UVC LEDs 82 and UVA LEDs 84, and also illumination by way of white-light LEDs 132 (or, in other embodiments, a white fluorescent tube, white incandescent bulb, or other white light source). This arrangement is beneficially compact. Although not shown in FIG. 10, it is contemplated for the electronics 86 to include the actinic dose budget parser 96 (and optionally sensors) operating as described with reference to FIG. 8.

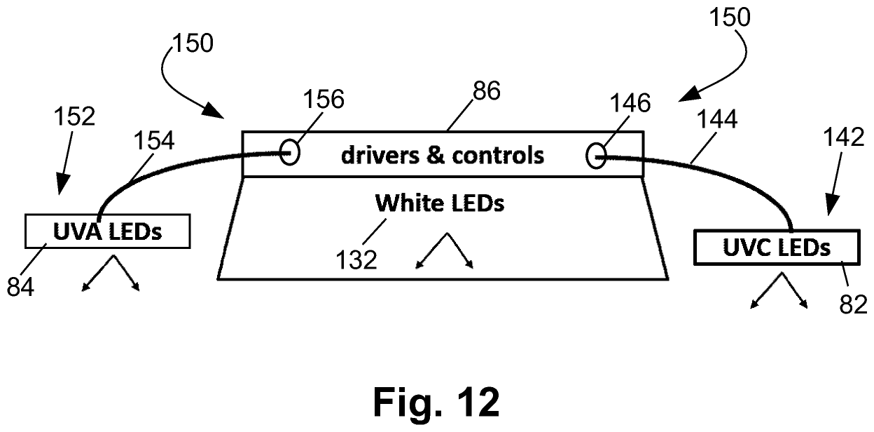

FIG. 11 illustrates an example similar to that of FIG. 10, except that in the example of FIG. 11 the UVA LEDs 84 and the white LEDs 132 are mounted in a main fixture 140 while the UVC LEDs 84 are mounted in an auxiliary fixture 142 connected with the driver and control electronics 86 by way of an electrical cable 144 connecting with a connector 146 of the main fixture 140. FIG. 12 illustrates an example similar to that of FIG. 11, except that here the main fixture 150 hosts only the white LEDs 132, with the UVC LEDs 84 again mounted in the auxiliary fixture 142 and here with the UVA LEDs 84 also mounted in an auxiliary fixture 152 which again is connected with the main fixture 150 by way of an electrical cable 154 connecting with a connector 156 of the main fixture 150. The arrangements of FIGS. 11 and 12 advantageously provide for modularity. For example, the main fixture 140 can be sold as a product and the auxiliary fixture or fixtures 142, 152 can be an optional add-on product(s).

Any of the fixture embodiments of FIGS. 8-12 may optionally include the actinic dose budget parser 96 to provide for adjusting the actinic dose budget between the UV LEDs of different wavelengths. Alternatively, any of the fixture embodiments of FIGS. 8-12 may omit the actinic dose budget parser 96, in which case the actinic dose fractions of the UV sources of the different wavelengths are fixed.

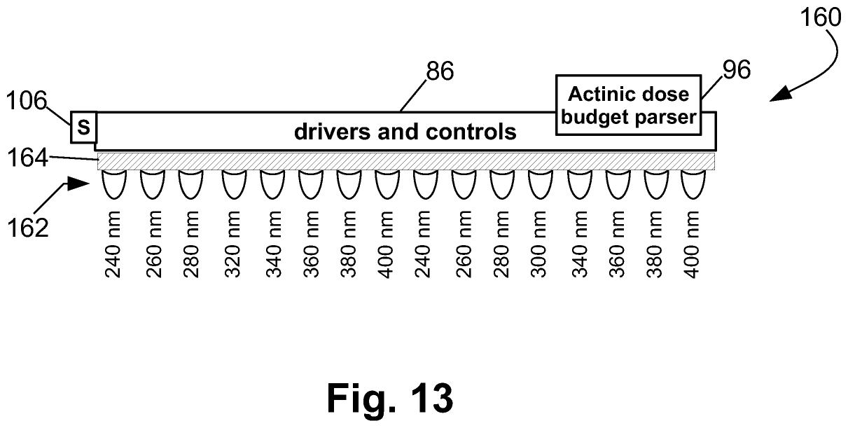

With reference to FIG. 13, UV LEDs enable near-exact selection of the inactivation wavelength for a given disinfection application. This is because LEDs are available with different peak wavelengths in about 5 nm increments, with about 10 nm linewidths (FWHM) throughout most of the UV, Visible, and Infrared regions of the electromagnetic spectrum. When combined with the actinic dose budget parser 96, in the embodiment of FIG. 13 this enables providing a light source for disinfection that provides a spectrum that is finely tailored for disinfecting a specific target pathogen. In the embodiment of FIG. 13, a single fixture 160 includes a bank of LEDs 162 with emission peaks at the labeled wavelengths in the (non-limiting illustrative) range of 240 nm to 400 nm inclusive in (non-limiting illustrative) 20 nm increments (except omitting an LED emitting at 300 nm which is in the UVB range), mounted on a PCB 164 with the drivers and controls electronics 86 including the actinic dose budget parser 96. In this embodiment, the spectrum can be tuned in 20 nm increments to match an experimentally determined optimal spectrum for inactivating a specific target viral or bacterial pathogen. For example, in the event of an outbreak of a specific pathogen, laboratory tests can be performed to optimize the UV spectrum for inactivating that specific pathogen. The actinic dose budge parser 96 is then set to energize the LEDs 162 of the various wavelengths to output actinic dose fractions in accord with (an approximation of) that empirically determined optimized UV spectrum, scaled in total dose to ensure the total dose remains below the actinic limit for safe occupation. (Optionally, if the fixture 160 further or is operatively connected with includes an occupancy sensor, then the output can be scaled up above the actinic limit for safe occupation when the space is determined to be unoccupied, as previously described with reference to FIG. 5). It will be appreciated that FIG. 13 is diagrammatic, and the LEDs of the various peak wavelengths may optionally be distributed in various ways over the two-dimensional area of the PCB 162. Moreover, while the illustrative fixture 160 contains LEDs 162 in the wavelength range 240-400 nm spanning large portions of the UV and violet spectral range, it is contemplated to include LEDs extending into other wavelength regions, such as the visible and infrared regions insofar as visible and infrared radiation can be effective for inactivating some types of pathogens.

As noted, the illustrative fixture 160 of FIG. 13 omits an LED emitting at 300 nm which is in the UVB range. This is based on the observation that light in the UVB range is typically less effective for inactivating pathogens, while having a high actinic hazard. Nonetheless, this is an illustrative example, and in some embodiments one or more of the LEDs may be emitting in the UVB range. The UVB range is especially efficacious in generating vitamin D in humans, and so UVB light sources may be included for this reason or for other reasons, although the UVB contribution to the 8-hour actinic dose would subtracted from the actinic dose allowed for inactivation of pathogens.

While reference is made to LEDs in describing the embodiments of FIGS. 8-13, it is to be appreciated that in some embodiments the LEDs may consist of a single LED, e.g. the UVC LEDs 82 may consist of a single UVC LED 82. Moreover, in other embodiments some or all of the LEDs may be replaced by other types of light sources (possibly including spectral filters) emitting at the design-basis wavelength peaks. For example, a low-pressure mercury lamp or an excimer lamp may be substituted for the UVC LEDs.

In the following, some contemplated multispectral UV disinfection light source embodiments are described in terms of some contemplated spectral components. In these examples, while a light source is referenced, it is to be understood the light source may be implemented by way of multiple fixtures, e.g. as in the examples of FIGS. 11 and 12.

In one illustrative embodiment, a multispectral light source includes a plurality of inactivating portions (or spectral regions), including a first inactivating portion having wavelengths in the UVA range and at least a second inactivating portion having wavelengths outside of the UVA range, e.g. a first inactivating portion having wavelengths in a range of about 315 nm to about 380 nm, and a second inactivating portion having wavelengths in a range below about 315 nm or in a range greater than about 380 nm, the accumulated actinic dose of the combined inactivating portions controlled to be below the exposure limit for human occupancy (e.g., the actinic UV hazard exposure limit for exposure to ultraviolet radiation incident upon the unprotected skin or eye apply to exposure within any 8-hour period). Note that in the Visible and Infrared regions, photobiological hazard limits other than actinic are applied. For example, the UVA limit for the eye pertains to wavelengths from about 315 to about 400 nm; the retinal blue light hazard pertains to wavelengths from about 400 to about 500 nm; the retinal thermal hazard pertains to wavelengths about 380 to about 1400 nm; and infrared eye hazard pertains to wavelengths about 780 to a bout 3000 nm. Although each of these other photobiological hazards, each having their respective action spectra and threshold limit values (or exposure limits) must be adhered to for a lighting system irradiating an occupied environment, typically only the actinic limit is of concern in designing a disinfection lighting system for pathogens, especially bacteria and viruses.

In another illustrative embodiment, a multispectral light source is configured to generate light in an environment for human occupancy, the light including a plurality of inactivating portions, including a first inactivating portion having wavelengths in the UVC range and at least a second inactivating portion having wavelengths outside of the UVA range, e.g. a first inactivating portion having wavelengths in a range of about 200 nm to about 280 nm, and a second inactivating portion having wavelengths in a range greater than about 280 nm, the accumulated actinic dose of the combined inactivating portions controlled to be below the exposure limit for human occupancy.

In another illustrative embodiment, a multispectral light source is configured to generate light in an environment for human occupancy that includes a plurality of inactivating portions, including a first inactivating portion having wavelengths in the UVA range and a second inactivating portion having wavelengths in the UVC range, and at least a third inactivating portion having wavelengths outside of the UVA and UVC ranges, e.g. a first inactivating portion having wavelengths in a range of about 315 nm to about 380 nm, and a second inactivating portion having wavelengths in a range of about 200 nm to about 280 nm and a third inactivating portion having wavelengths in a range greater than about 380 nm or between about 280 nm and about 315 nm (or, in some alternative embodiments, 280-320 nm), the accumulated actinic dose of the combined inactivating portions controlled to be below the exposure limit for human occupancy.

In another illustrative embodiment, a multispectral light source is configured to generate light in an environment for human occupancy that includes a first inactivating portion having wavelengths in the deep UVC (or far-UV) range (e.g. about 240 nm or lower, or more preferably 225 nm or lower in some non-limiting illustrative embodiments, e.g. at about 222 nm in some non-limiting illustrative embodiments) that is inactivating for a target pathogen, and a second UV portion in the range about 240 nm or longer, which may or may not be inactivating for the target pathogen. The term deep-UV or far-UV is not well defined in the lighting arts, therefore herein it is defined to be any wavelength shorter than about 242 nm, which is the onset below which ozone may be generated in the air (which can create human health concerns, certainly so in pulmonary-compromised individuals). An advantage of this spectrum is as follows. The shorter wavelength (242 nm or lower) has a disadvantage of generating ozone. However, the second UV portion having a longer wavelength e.g., in the range about 242 nm or longer operates to decompose the ozone, thereby allowing for reduced ozone emission of the overall multispectral light source while still providing shorter wavelength (242 nm or lower) emission for inactivating the target pathogen. A further advantage of this multispectral combination is that the actinic weighting at 242 nm and shorter is relatively low, e.g., about 20% or less than at some longer UV wavelengths, while still providing significant inactivation of pathogens, so that a substantial portion of the 8-hour actinic limit may be reserved for removal of ozone by the longer UV wavelengths having a higher actinic weighting.

In further illustrative embodiments, a multispectral light source configured to generate light in an environment for human occupancy includes three or more inactivating portions.

In some illustrative embodiments, a multispectral light source configured to generate light in an environment for human occupancy emits light in two or more discrete peaks, for example corresponding to UVA LEDs emitting at a peak in the UVA spectrum (315 nm to 400 nm inclusive) and UVC LEDs emitting at a peak in the UVC spectrum (100 nm to 280 nm inclusive), and optionally further including one or more additional LEDs such as violet LEDs emitting at a peak in the violet spectrum (380 nm to 450 nm inclusive). In some non-limiting illustrative embodiments, the total emission intensity of the multispectral light source outside of these two or more discrete peaks is less than 40% of the total intensity emitted by the multispectral light source.

With continuing reference to the embodiment of FIG. 13, in which the fixture 160 includes a set of LEDs in the range 240-400 nm, optimization of the spectrum can be performed as follows. Each wavelength has a corresponding actinic hazard coefficient k_{act}, for example also taken from the published ACGIH 42671 action spectrum. Then the actinic dose is given by:

$${D}_{act} = {\sum }_{i = 1}^{N} {k}_{act , i} {H}_{i} < actinic limit , EL \qquad ( 5 )$$

where Hᵢ denotes the “radiant exposure” or “dose” [J/m²] of the LEDs indexed by index i. As indicated in Equation (5), the design must keep this actinic dose D_{act} below the actinic limit, EL=30 J/m². On the other hand, the germicidal efficacy, E_{germ}, can be expressed as:

$${E}_{germ} = {\sum }_{i = 1}^{N} {k}_{germ} {H}_{i} \qquad ( 6 )$$

where again k_{germ,i} is the germicidal coefficient for the LEDs indexed by i. Hence, it is desirable to maximize the germicidal efficacy, E_{germ}, for the specific pathogen by maximizing Equation (6) while ensuring the constraint D_{act}<actinic limit, EL as set forth in Equation (5) is satisfied. The actinic dose budget parser 96 suitably does this by adjusting the radiant exposures or doses Hᵢ, i=1, . . . , N for example using a least squares optimization (e.g., Levenberg-Marquardt algorithm). The spectral distribution (which in some cases may be a set of discrete wavelength) may then be designed to optimally inactivate a specific target pathogen, for example based on Equation (6) using the germicidal efficacy E_{germ} as a function of wavelength determined experimentally for the specific target pathogen, with the amplitude of the designed spectrum then being adjusted to obey the actinic limit set forth in Equation (5).

While not illustrated in FIGS. 2 and 3, it will be appreciated that the light sources of FIGS. 2 and 3 may optionally include an electronic processor (e.g., a microprocessor or microcontroller) analogous to the driver and control electronics 86 of the embodiments of FIGS. 8-13, which is programmed to control the actinic light output by the light source. If the light source outputs a single wavelength or spectrum (e.g. the gas discharge lamp of FIG. 3), then the driver and control electronics may control the actinic light output in a binary fashion (on or off) or by a continuous or stepwise intensity adjustment.

It will be further appreciated that any of the light sources of FIGS. 2, 3, 8, 9, 10, 11, 12, and/or 13 may in some embodiments have only sufficient drivers and controls 86 to receive a control signal from the optional network controller 69. In these embodiments, control logic is implemented at the network controller 69, which then sends control signals (e.g. signals indicating a light intensity or LED voltage or the like) to the light sources of the light source communication network 68. In these embodiments, the driver and control electronics of each individual light source 10 is limited to driver electronics including hardware sufficient to receive the control signal from the network controller 69 and to control the driver to output the actinic light in accord with that received control signal.

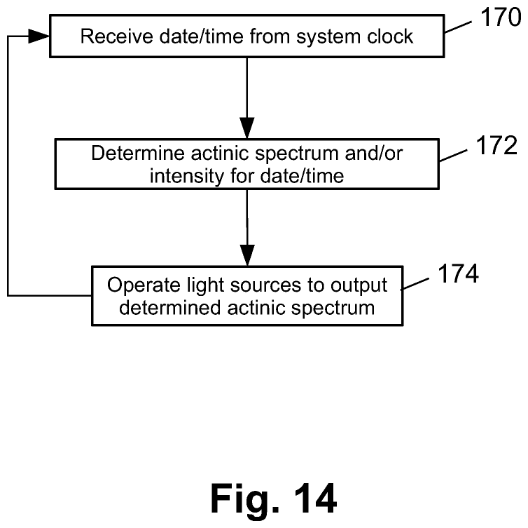

With reference to FIG. 14, the actinic spectrum output by the multispectral light source 160 of FIG. 13 may in some embodiments be adjusted automatically on the basis of the current date/time. In an operation 170, the current date/time is received from a system clock of the network controller 69 (or, alternatively, a clock of the on-board driver and control electronics 86 of a light source of FIGS. 8-13; more generally, a clock of or accessible by the electronic processor providing the lighting control). In an operation 172, the network controller 69 determines the appropriate actinic spectrum for the date/time received at operation 170. In an operation 174 the multispectral light source(s) 160 are operated to output the determined actinic spectrum. The operations 170, 172, 174 may be repeated in a loop to optimize the actinic spectrum output by the light source 160 for conditions present at specific dates or times. The control of FIG. 14 may be implemented in the driver and controls 86 of the multispectral light source 160 of FIG. 13, or as part of a lighting control software package running on the network controller 69 operatively connected to the light source 160 by way of the light source communication network 68.

For example, the control process of FIG. 14 can be used to perform seasonal adjustment of the actinic spectrum. It may be known that certain pathogens are more prevalent during certain seasons of year (e.g. spring, summer, autumn, or winter), and that other pathogens are more prevalent during other seasons. Furthermore, different pathogen-optimized actinic spectra may be available for these different pathogens. The different pathogen-optimized actinic spectra may be predetermined from pathogen-specific laboratory experiments, and/or from first principles (e.g., UVC is generally more efficacious for viral pathogens while UVA is generally more efficacious for bacterial pathogens). Given such information, a look-up table can be generated that associates different seasons with different actinic spectra, and the operation 172 then references the look-up table to retrieve the appropriate actinic spectrum for the date retrieved at the operation 170. As a more specific example of seasonal adjustment, if the environment 2 is an athletic locker room then the dominant pathogen may be mold pathogens throughout the year. However, during winter flu season, influenza viral pathogens may be of greater concern than mold pathogens. Hence, a UVA-based actinic spectrum optimized for inactivating mold pathogens may be applied during the spring, summer, and autumn seasons, while a UVC-based actinic spectrum optimized for inactivating influenza may be applied during the winter flu season. This is merely an illustrative example; in other examples, the spectral output may be adjusted with temporal granularity on the order of days or hours.

With continuing reference to FIG. 14, in another embodiment, in the operation 172 the network controller 69 determines the appropriate intensity for the date/time received at operation 170. For example, the intensity may be turned down or off at certain times of day based on expected occupancy, or to provide a desired time schedule of disinfection (e.g., 3 hours of disinfection overnight).

In another contemplated embodiment, the multispectral light source 160 of FIG. 13 may incorporate one or more sensor 106 (already described with reference to FIG. 8). For example, the sensor(s) 106 of (or operative in conjunction with) the multispectral light source 160 may detect specific pathogens and tune the spectrum of the light output by the multispectral light source 160 to specifically target the detected pathogens, using stored spectra optimized for various pathogens of interest for inactivation. For example, Hospital-Acquired-Infections (HAIs) are a result of a number of (primarily bacterial) species that spread between patients as a result of imperfect sanitization processes (e.g. cleaning of hands, equipment, surfaces, etc.). It is also well-known that many of these infectious species have acquired ‘immunity’ to a wide range of antibiotics (typically by overuse), perhaps best exemplified by MRSA: Methicillin-resistant Staphylococcus aureus, but include a growing list of other Pathogens. Hence, if the biosensor 106 detects a known HAI species then the spectrum of light output by the light source 160 can be a principally UVA light spectrum that is tailored for that species. As other examples, many viral outbreaks are passed human-to-human, either via direct-contact or via droplets, aerosols, fomites, or so forth. Example Pathogens that cause societal havoc, either sporadically or seasonally, include: corona-viruses (SARS/MERS/SARS-CoV-2); various strains of Influenzas; pneumonia; tuberculosis; and so forth. Hence, if the biosensor 106 detects one of these viruses then the spectrum of light output by the light source 160 can be a principally UVC light spectrum that is tailored for that species. As yet other possible target pathogens, cruise-ships and commercial airliners have become locations for inter-passenger disease communicability (e.g. Norovirus); while, certain work/living environments (e.g. nursing homes, meat-processing plants, prisons) place humans into unavoidably close quarters, becoming noted ‘breakout’ loci for epidemics, both viral and bacterial, so that biosensor-based tuning of the disinfection light can be advantageous in these settings as well. Each of these (non-limiting illustrative) pathogens has a different ‘log kill-rate’ at the different available UV wavelengths or spectra (see, e.g. Kowalski 2009). It is also contemplated to adjust the overall intensity based on the sensor reading of the biosensor 106. For example, if the biosensor 106 detects a particularly contagious or particularly dangerous pathogen then the intensity may be increased above its usual level as the danger posed by the pathogen may be substantially greater than the danger posed by the increased UV dose.

If biosensors are not incorporated into the light source to provide automated spectral tuning to target specific pathogens, then this could be done manually. The occurrence of different pathogenic species typically varies over time, e.g. a regional or larger-scale epidemic may bring large numbers of infectious patents with a specific pathogen into a hospital for a few weeks or months, only to be supplanted by some more recent outbreak. Hospitals conduct periodic mandated sanitary audits (swabs and assays) of surfaces in the medical setting that show results that may vary, month to month. The network controller 69 may have look-up tables with different spectra for different pathogens, and then by merely selecting the dominant pathogen indicated by the sanitary audit the network controller 69 tunes the spectrum produced by the multispectral light source 160 to that pathogen.

In a variant embodiment, if the sanitary audit or biosensors indicate two (or possibly more) dominant pathogens, the network controller 69 may tune the spectrum produced by the multispectral light source 160 to a weighted combination of the optimized spectra for those two (or more) pathogens. The weighting can be chosen, e.g. based on the relative abundances of the two (or more) dominant pathogens, and/or their perceived severity (e.g., biasing toward inactivation of the more dangerous pathogen).

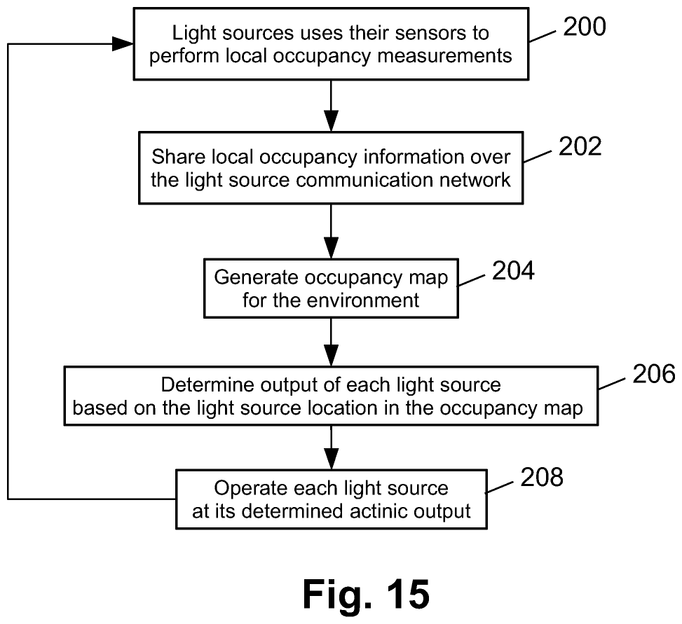

With returning reference to FIG. 1 and with further reference to FIG. 15, an embodiment which leverages the light source communication network 68 is described. In this embodiment the environment 2 is relatively large (for example, a large warehouse), and the light source communication network 68 is designed to locally operate individual light sources or groups of light sources at the higher intensity based on local occupancy. In an operation 200, the light sources 10 use their sensors 40, 42 (see FIGS. 2 and 3) to perform occupancy measurements in their respective local areas. In an operation 202, these occupancy measurements are shared over the light source communication network 68. This sharing may be amongst the light sources 10, or may be shared to the optional network controller 69. In an operation 204, the occupancy data from all occupancy sensors of the light source communication network 68 are combined to generate an occupancy map with lateral resolution comparable to the lateral spacing of the light sources 10 over the environment 2. The occupancy mapping operation 204 may be performed at each light source 10 if the individual light sources have sufficient computing capacity; or, the occupancy mapping operation 204 may be performed at the network controller 69 (if provided) and then the occupancy map is distributed to the individual light sources 10 over the light source communication network 68. Each light source 10 is further programmed with its location in the environment 2 and with an effective coverage area denoting the spatial range over which light from that light source is received. With this information (the occupancy map and the light source-specific location and light spread), in an operation 206 performed at each light source 10, the light source independently decides in real-time whether to operate at the higher intensity, lower intensity, or be turned off, e.g. in accord with a control paradigm such as one of those depicted in FIG. 5, based on whether the area of light spread of that light source is currently unoccupied in the occupancy map. In a variant approach, the network controller 69 is provided with the location of each light source 10 in the environment 2, and so the network controller 69 can perform the operation 206 for each light source 10 and then sends (via the network 68) a respective control signal to each light source 10 based on the determination at the network controller 69 of whether the light source 10 should be operating at the higher intensity. Finally, in an operation 208 each light source 10 operates at the determined actinic output. The operations 200, 202, 204, 206, 208 can be rapidly repeated, e.g. once every ten seconds or faster, and in some embodiments more preferably every second or faster, to cause the distribution of actinic output to track movement of individuals in real time. By “real time” in this context it is meant that the updating via repetition of the operations 200, 202, 204, 206, 208 is sufficiently fast to track movement of human occupants in the environment 2.

The control approach of FIG. 15 is particularly advantageous if the warehouse or other large environment 2 is always or usually occupied during a work shift, but relatively sparsely. In this case, at any given time most of the light sources 10 will have their beam spread entirely unoccupied and hence can be operating at the higher intensity; while, those light sources 10 whose beam spread is currently occupied are turned to a lower intensity (below the actinic dose exposure limit for the dose time period). Optionally, light sources 10 whose beam spread is currently entirely unoccupied may be operated at an intensity that is such that the dose would exceed the actinic dose limit over 24 hours (or other dose time period for which the actinic dose limit is defined), since there is no occupant receiving that ultraviolet light. Preferably, safety margins are built into this arrangement, e.g. (the mathematical representation of) the light spread used in the decision-making operation 206 may be set to be, e.g., 50% larger than the actual light spread to provide some safety margin. In this way, for a large environment that is sparsely occupied, some (possibly most) of the light sources 10 will be operating at the higher intensity at any given time, thereby increasing disinfection efficacy. If the large environment 2 also has efficient air circulation provided by large ceiling fans and/or a building-wide HVAC system, then even the areas that are currently occupied at any given time will experience heightened disinfection efficacy as air circulates between the occupied areas that are not being actively disinfected and neighboring unoccupied areas that are being actively disinfected. However, even in a small space, such as an office, the control approach of FIG. 15 can be useful. For example, the office may have as few as two or three light sources 10 and the approach of FIG. 15 can be used to operate the light source(s) covering any unoccupied area(s) of the environment 2 (e.g. the office) at high intensity and the light source(s) covering any occupied area(s) of the environment 2 (e.g. the office) at lower intensity (or zero intensity, i.e. turned off).

In some embodiments employing the control approach of FIG. 15, the light sources 10 may be designed to output the ultraviolet light with a relatively narrow beam, so that the ultraviolet light output by each light source 10 is a column of light, i.e. a narrow beam of light with a small angle of divergence. A narrow beam of light can be achieved using an ultraviolet LED, excimer lamp, laser diode, or so forth coupled with a parabolic or other directional reflector and/or a converging lens made of an ultraviolet-transmissive material such as sapphire, fused quartz, UV-transmissive silicone, single-crystal aluminum oxide, or so forth. (In the case of an inherently relatively collimated source such as a laser diode, the optic may optionally be omitted). With reference back to FIG. 1, in this design the ceiling-mounted light sources 10 output mutually parallel vertically oriented beams, with the beam output by each light source 10 projecting downward toward the floor 6. The beams optionally may not overlap, if air circulation in the environment 2 is sufficient to efficiently move air between the ultraviolet light beams generated by the light sources 10. Advantageously, the small angle of divergence of the ultraviolet light beams means that each beam has a small and well-defined light spread, which simplifies assessment of whether the area of light spread of each light source 10 is currently unoccupied in the occupancy map. Due to the narrow light spread, all of the light sources 10 can be operating except those light source(s) that are directly (or almost directly) above an occupant as indicated by the occupancy map. Moreover, the small and well-defined light spread of the ultraviolet light beams means that even if an occupant is standing next to a light beam, that occupant will receive little ultraviolet light dosage from the light beam so long as the occupant is not within the beam. Consequently, it is contemplated to operate the operating light sources 10 (those not directly or almost directly overhead of an occupant) at high intensity, that is, at an intensity that exceeds the intensity that would produce a dose exceeding the actinic dose limit if received over the design-basis dose time period. Those light sources that are (almost) directly overhead of an occupant such that the occupant would impinge on the beam are turned off (or at least are lowered to an intensity for which the dose over the design-basis dose limit is within the actinic dose limit). Advantageously, if the environment 2 has reasonably sparse occupancy, then this means that at any given time most of the light sources 10 are operating to provide disinfection, and indeed are operating at high intensity that exceeds the intensity that would produce a dose exceeding the actinic dose limit if received over the design-basis dose time period.

In a variant embodiment employing the control approach of FIG. 15, in the decision-making operation 206 the network controller 69 determines one or more additional operational parameters for each light source 10 of the plurality of light sources. The one or more additional operational parameters for each light source 10 is in addition to the intensity for each light source. In the control operation 208, the control of each light source 10 is then (in addition to being at the determined intensity) also in accord with the one or more additional operational parameters determined for that light source.

For example, the one or more additional operational parameters could include a geometric beam parameter such as an ultraviolet light beam width or an ultraviolet light beam direction parameter. As an example of the latter, each light source 10 can output in two or more different directions, with each beam direction being provided by one or more LEDs oriented to emit light in that direction. (Another way directionality could be achieved is by having the LEDs of the light source 10 mounted on a gimbal mount or other type of mount by which the LEDs can be mechanically tilted to achieve different directions). Here the network controller 69 in the decision-making operation 206 uses the occupancy map to select beam directions that direct the ultraviolet light beams away from any occupied areas and toward unoccupied areas.

As another example, if the light sources 10 are multispectral (e.g. as in the example of FIG. 13) then the one or more additional operational parameters may include one or more spectral parameters. For example, light sources delivering ultraviolet light to occupied areas can be spectrally adjusted to a wavelength or spectral peak that presents a lower actinic hazard (possibly at the cost of being less effective for inactivating a target pathogen); while, light sources delivering ultraviolet light to unoccupied areas can be spectrally adjusted to a wavelength or spectral peak that is highly effective for inactivating the target pathogen (even if this presents a higher actinic hazard).

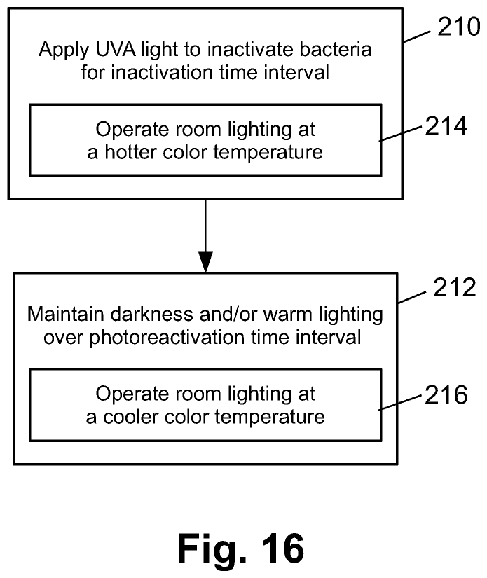

With reference to FIG. 16, embodiments are described which combat photoreactivation. As previously mentioned, some bacteria can repair genetic damage caused by ultraviolet light by action of photolyases or other DNA repair enzymes, a process known as photoreactivation. However, photolyases are themselves light activated, typically by light in the blue or violet spectral range. Hence, an optimal disinfection sequence for such bacteria shown in FIG. 16 (suitably implemented by programming the electronic controllers of the light sources 10 or the network controller 69) includes an operation 210 of applying UVA light to the environment 2 to inactivate the bacteria, followed by an operation 212 comprising a period of darkness of the environment 2 to prevent photoreactivation of the damaged bacterial DNA.

Alternatively, if the environment 2 may need to be illuminated with room light during the time period of the operation 212, then the operation 212 may include operating the room lighting at a lower color temperature in order to reduce the blue and violet spectral components and thereby reduce or eliminate photoreactivation. Typical indoor white lighting may be categorized as: soft white (color temperature 2700-3000 K); warm white (color temperature 3000-4000 K); bright white (color temperature 4000-5000 K); or daylight (color temperature 5000-6500 K). A higher color temperature given in Kelvin (K) in the foregoing list corresponds to the white light having a higher fraction of shorter wavelength (e.g. blue) content, while a lower color temperature corresponds to a higher fraction of longer wavelength (e.g. red) content. Hence, for example, if the normal white light has a color temperature of greater than 3000K (typically used in indoor settings such as bathrooms, kitchens, offices, et cetera; in some alternative embodiments may be greater than 2700K or some other lower end limit) then during the time period of the operation 210 if the room lights are on then they are operated at a higher color temperature, e.g. at a color temperature of greater than 3000K (operation 214); whereas, if the room lights are operated during the time period of the operation 212 after the UVA application then the room lights are operated at a lower color temperature, e.g. at a color temperature of 3000K or lower (operation 216). (Alternatively, the blue light content could be reduced while maintaining the color temperature by adjusting the spectral shape). For occupants of the room or other environment 2, the operation 214 will be experienced as “normal” warm white, bright white, or daylight-level lighting. The operation 216, on the other hand, will be experienced as a more “reddish” warm white or soft white lighting. It may be more preferable for the lighting during operation 216 to be nearly or completely free of blue light, e.g. 2100 K or 1900 K or the like.

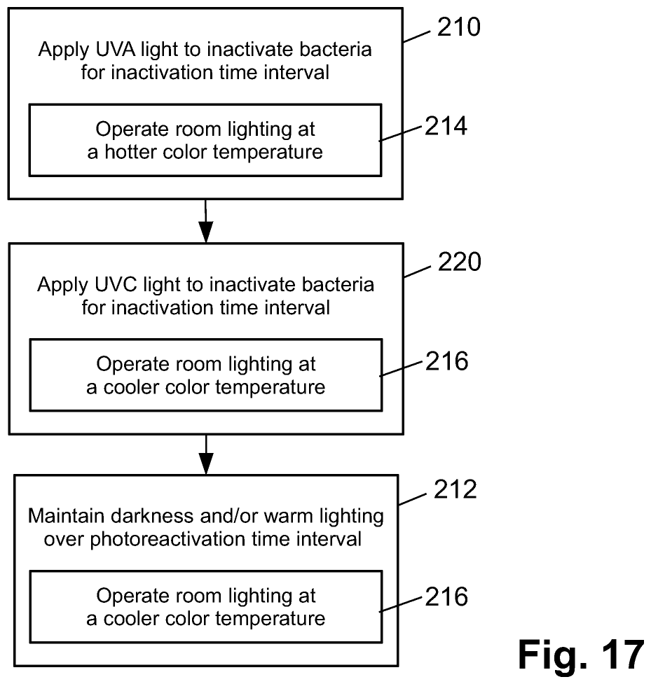

With reference to FIG. 17, a variant approach of FIG. 16 is shown which is suitably employed if both UVA light and UVC light are to be applied. For example, the UVA light is suitably applied to inactivate bacterial pathogens and the UVC light is suitably applied to inactivate viral pathogens. The approach of FIG. 17 recognizes that UVC light is generally not effective for driving photoreactivation in bacteria. Hence, in the example of FIG. 17, in an operation 220 UVC light is applied for a time period after the UVA is applied. The UVC applied in the operation 220 does not promote photoreactivation of the bacteria inactivated in the prior operation 210. During the UVC operation 220, room lighting (if used) is preferably controlled during the time period of the UVC operation 220 the same way as in the subsequent operation 212, i.e. if the room lights are operated during the time period of the UVC operation 220 after the UVA application then the room lights are operated at the cooler color temperature, e.g. at a color temperature of 3000K or lower (operation 216).

In a variant of the approach of FIG. 17, the time interval of the UVA application 210 and the time interval of the UVC application 220 could partially or entirely overlap in time. In such a variant approach, the operation 214 (room lighting operating >3000K) could be performed until the UVA light is turned off, and thereafter if room lighting is used it would be at the lower color temperature (operation 216).

To implement the operation 216 of FIGS. 16 and 17 in which the color temperature of the room lighting is lowered, the (white) room lighting should be under control of the disinfection system. For example, white LED light sources providing the room lighting may be included in the light source communication network 68 and thereby be under control of the network controller 69, which is then programmed to implement the color temperature shift of operation 216 as appropriate to suppress photoreactivation of bacteria subsequent to the UVA operation 210.

The disclosed disinfection control approaches are generally usable with any of the light sources of FIG. 2, 3, or 8-13. For disclosed control systems employing an electronic processor (e.g. microprocessor or microcontroller), the electronic processor may be implemented: on-board the light source (e.g., as the driver and controls 86 of the illustrative embodiments of FIGS. 8-13); in a network controller 69 in conjunction with a light source communication network 68; or as a combination thereof (e.g., implementing control operations involving complex processing and/or utilizing extensive memory to store pathogen-specific spectra or the like by an electronic processor of the network controller 69 and implementing less computational- and memory-intensive control operations by an on-board electronic processor of the light source(s)).

Furthermore, it will be appreciated that the disclosed disinfection control approaches can be embodied as a non-transitory storage medium storing instructions that are readable and executable by the electronic processor(s) to perform the disclosed disinfection control approaches. The non-transitory storage medium may, for example, comprise: a programmable read-only memory (PROM), electronically erasable programmable read-only memory (EEPROM), a flash memory or other electronic non-transitory storage medium; a hard disk or other magnetic storage medium; an optical disk or other optical storage medium; various combinations thereof; or so forth. As previously noted, if the network controller 69 is provided then it may include a display for presenting sensor readings, light source configuration data, and/or so forth, and the network controller 69 may further include a keyboard, mouse, touch-sensitive display, and/or other user input device(s) via which a user can configure the light sources 10 as disclosed herein.

On the other hand, as further disclosed herein, in some embodiments the light source communication network 68 and network controller 69 may be omitted, and each light source 10 may be independently configurable by way of (for example) a dial, slider or other user input for setting the intensity, manual switches for setting control settings such as the light source-to-head level distance, and/or so forth.

Because UV exposure limits in an occupied (or possibly occupied) environment implicates safety, it can be useful to have multiple partly or wholly redundant interlocks to ensure that the actinic dose limit is not exceeded.

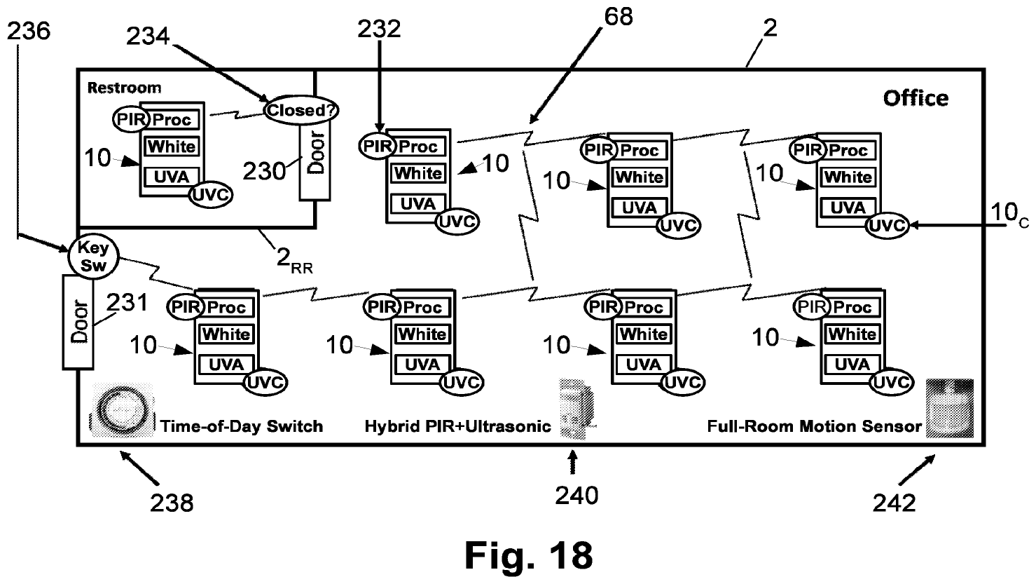

With reference to FIG. 18, an illustrative example is shown in which the environment 2 for human occupancy is a diagrammatically shown office, and further includes an environment 2_{RR} for human occupancy which is a diagrammatically shown restroom. The restroom 2_{RR} is accessible from the office 2 via a door 230, and access to the office 2 is controlled by an entry door 231. In this embodiment the light sources 10 are combined white/UVA/UVC light sources in which the UVC light source is mounted as an auxiliary fixture 10c (e.g., an example of which was previously described with reference to FIG. 11). The light sources 10 are wirelessly interconnected via the light source communication network 68 as previously described. The light sources 10 include embedded occupancy sensors 232, such as passive infrared (PIR) motion sensors, as already described (see motion sensors 40 of the light source 10 of FIG. 2). These occupancy sensors 232 provide a first level of control to limit actinic dose. For example, the light sources 10 may operate at intensities producing UV exposure above the actinic dose limit for the dose time period (e.g., an 8-hour workday); except that when the occupancy sensors 232 detect an occupant in the environment 2 or 2_{RR} then the UV output of the light sources 10 is turned off or lowered in intensity to be below the intensities producing UV exposure above the actinic dose limit for the dose time period. Such operation has been previously described, for example, with reference to the right-hand flowchart of FIG. 5.

To provide further safety against overexposure of ultraviolet light to occupants, additional hard-wired cutoffs or control electronics 234, 236, 238 may be provided. For example, if the restroom door 230 is normally open and is closed only when in use (for example, a swinging door that has a latch that is engaged from the inside to lock the door 230 from the inside), then it is very likely that the restroom 2_{RR} is unoccupied if the door 230 is open and is occupied if the door 230 is closed. Hence, a door-closed sensor 234 may detect when the door 230 is closed and turn off (or lower the intensity of) the light source 10 in the restroom 2_{RR} when the door 230 is detected to be closed. (In a variant embodiment, the intensity may be off or low when the door is opened and raised to a higher intensity level when the door is closed and perhaps for some fixed time interval after the door re-opens, analogous to the approach of FIG. 5, left-hand flowchart). In a similar fashion, a key switch 236 of the entryway door 231 of the office 2 can serve as a sensor. Here, the office 2 is assumed to be unoccupied if the key switch 236 is locked. The workflow is assumed to be that the first person into the office (or, in a variant, a security guard) unlocks the key switch 236 at the start of the workday, and the last person out (or the security guard) locks the key switch 236 at the end of the day. Hence, the key switch 236 operates to turn off (or lower the intensity of) the light sources 10 in the office 2 when the key switch 236 is detected to be unlocked. As yet another example, a time-of-day switch or clock 238 can be used to turn off (or lower the intensity of) the light sources 10 in the office 2 when the time of day as measured by the clock 238 is in normal working hours. More generally, electronics of the light source or lighting system may be configured to control the intensity and/or spectrum of the ultraviolet light emitted by the one or more light sources based on a date and/or time provided by the clock 238. These various cutoffs or control electronics 234, 236, 238 suitably operate independently of the control provided by the occupancy sensors 232 of the light sources 10, although they may use the light source communication network 68 to provide the sensor output to the light sources 10 (or to the network controller 69, see FIG. 1, if that controller 69 controls operation of the light sources 10).

Moreover, it will be appreciated that in some embodiments the various cutoffs or control electronics 234, 236, 238 may be implemented as analog or digital electronics that do not include a microprocessor, microcontroller or other electronic controller. For example, a cutoff can be implemented as an analog switch that interrupts the electrical current; while a more complex control paradigm such as the having the light source(s) stay on or off for a predetermined time interval after triggering can be implemented by triggering the current interruption switch off of an analog or digital delay timer with the delay time hard-wired by way of a timer circuit capacitance or the like. A control paradigm in which the intensity is lowered or raised over a time interval can be similarly implemented by switching a voltage divider in or out. These are merely non-limiting illustrative examples.

With continuing reference to FIG. 18, in a variant embodiment (optionally used in combination with other embodiments described with reference to FIG. 18), one or more cutoff control devices may be provided that operate independently of the light source communication network 68. For example, the time-of-day switch or clock 238 may instead be hard-wired to an AC power circuit driving the light sources 10 (or, in a variant embodiment, driving the UV components of the light sources 10 but not the white light components of the light sources 10). In this case, the time-of-day switch or clock 238 interrupts AC power to the light sources 10 (or to the UV components of those light sources) when the time of day as measured by the clock 238 is in normal working hours. Similarly, a hybrid PIR and ultrasonic sensor 240 or a full-room motion sensor 242 may be hard-wired to the AC power circuit driving the light sources 10 (or driving the UV components thereof) so as to interrupt AC power to the light sources 10 (or to the UV components thereof) when the sensor 240, 242 detects motion in the office 2. An advantage of this approach is that it provides an independent safety interlock in the event that the light source communication network 68 fails to shut off or lower intensity of the light sources 10.

Other approaches can be employed to provide independent safety interlocks. For example, the light sources 10 can be programmed to turn off the UV light emission in the event that contact with the light source communication network 68 is lost. In other words, the light sources 10 are “normally off” (at least as far as UV emission) and only emit UV light when they are in contact with the light source communication network 68 and the light source communication network 68 is sending a signal to operate to emit UV light.

The present disclosure has been described with reference to exemplary embodiments. Modifications and alterations may occur to others upon reading and understanding the preceding detailed description. It is intended that the present disclosure be construed as including all such modifications and alterations insofar as they come within the scope of the appended claims or the equivalents thereof.

1. A disinfection system comprising:
one or more light sources configured to emit light including at least ultraviolet light effective for inactivating pathogens in an environment for human occupancy;
one or more sensors configured to acquire data indicative of the environment for human occupancy; and
electronics included or operatively connected with the one or more light sources and configured to control an intensity and/or spectrum of the ultraviolet light emitted by the one or more light sources based on the data indicative of the environment acquired by the one or more sensors.

2. The disinfection system of claim 1 wherein the one or more sensors include one or more of: a motion sensor, a microphone, a bed weight sensor, a proximity or distance sensor secured with the light source, electronic entry security device implemented at an accessway of the environment for human occupancy, and/or an RFID badge reader.

3. The disinfection system of claim 1 wherein the one or more sensors include a microphone, and the electronics are configured to increase the ultraviolet light emitted by the plurality of light sources in response to detection by the microphone of a sound indicative of an aerosol emission event produced by an occupant of the environment for human occupancy.

4. The disinfection system of claim 1 wherein the one or more sensors include a proximity or distance sensor secured with the light source, and the electronics are configured to reduce or turn off the intensity of the ultraviolet light emitted by the one or more light sources in response to the proximity or distance sensor detecting an object closer than a programmed or fixed distance.

5. The disinfection system of claim 1 wherein the one or more sensors include a proximity or distance sensor secured with the light source, and the electronics are configured to reduce or turn off the intensity of the ultraviolet light emitted by the one or more light sources in response to an object proximity measured by the proximity or distance sensor.

6. The disinfection system of claim 1 wherein the one or more light sources are configured to emit the ultraviolet light effective for inactivating pathogens in the environment for human occupancy with a configurable spectrum, and the electronics are further configured to control the spectrum of the ultraviolet light emitted by the one or more light sources.

7-8. (canceled)

9. The disinfection system of claim 1 wherein the electronics are configured to control the intensity and/or spectrum of the ultraviolet light emitted by the one or more light sources further based on a date and/or time provided by a clock of or accessible by the electronics.

10. (canceled)

11. The disinfection system of claim 1 wherein the one or more light sources comprise a plurality of light sources configured as a light source communication network, and the electronics are configured to control the intensity of the ultraviolet light emitted by the plurality of light sources via the light source communication network.

12. The disinfection system of claim 11 wherein the electronics includes a user input device and are further configured to control the intensity of the ultraviolet light emitted by the plurality of light sources based on a dose time period received via the user input device.

13. The disinfection system of claim 11 wherein the electronics are further configured to:
control the plurality of light sources to emit UVA light over a first time interval followed by a second time interval during which UVA light is not emitted; and
control room lighting of the environment for human occupancy during the second time interval to emit white light at a color temperature of 3000K or lower.

14. (canceled)

15. The disinfection system of claim 11 wherein the electronics comprise an electronic processor programmed to control the intensity of the ultraviolet light emitted by the plurality of light sources by operations including:
generating an occupancy map of the environment for human occupancy using the data indicative of the environment acquired by the one or more sensors;
determining an intensity for each light source of the plurality of light sources based on the occupancy map and locations of the light sources in the occupancy map; and
controlling each light source of the plurality of light sources to emit ultraviolet light at the intensity determined for that light source.

16. The disinfection system of claim 1 further comprising:
dosimeters worn by occupants of the environment;
wherein the electronics are configured to control the intensity of the ultraviolet light emitted by the one or more light sources further based on ultraviolet doses received by the occupants as determined by the dosimeters.

17. The disinfection system of claim 1 wherein:
the one or more sensors comprise identification badges worn by occupants; and
the electronics are configured to control the intensity of the ultraviolet light emitted by the one or more light sources based on ultraviolet doses received by occupants determined from the data indicative of the environment including ultraviolet doses received by the occupants computed based on tracking of the occupants using the identification badges.

18-19. (canceled)

20. A method of configuring a light source to be used for disinfecting an environment for human occupancy, the method comprising:
determining a light source-to-head level distance for the environment;
determining a maximum permissible irradiance for safe occupation based on a dose time period and an actinic dose limit;
determining a light source intensity for the light source based on the light source-to-head level distance and the maximum permissible irradiance for safe occupation; and
operating an intensity setting input of the light source to adjust an intensity output by the light source to the determined irradiance.

21. The method of claim 20 further comprising using a photosensor to measure the intensity output by the light source during or after the operating.

22. The method of claim 20 further wherein the determining of the light source intensity is further based on a scaling factor correcting for overlap of light from one or more neighboring light sources.

23. The method of claim 20 further comprising labeling the light source with a warning label indicating the light source-to-head level distance as a minimum light source-to-head level distance.

24-28. (canceled)

29. A disinfection system comprising:
a plurality of light sources distributed in an environment for human occupancy and configured to emit light including at least ultraviolet light;
sensors distributed in the environment for human occupancy and configured to acquire data indicative of real-time spatially resolved occupancy of the environment for human occupancy; and
at least one electronic processor operatively connected with the light sources and the sensors and programmed to:
generate an occupancy map of the environment for human occupancy using the data indicative of real-time spatially resolved occupancy;
determine intensities for respective light sources of the plurality of light sources based on the occupancy map and locations of the light sources of the plurality of light sources in the occupancy map; and
control the respective light sources of the plurality of light sources to emit light at the intensity determined for that light source.

30. The disinfection system of claim 29 wherein the electronic processor is further configured to:
determine one or more additional operational parameters for the respective light sources of the plurality of light sources wherein the one or more additional operational parameters is in addition to the intensity for each light source, and
wherein the control of the respective light sources is in accord with the one or more additional operational parameters determined for the respective light sources and the one or more additional operational parameters includes a geometric beam parameter and/or a parameter defining a spectrum of the light.

31-32. (canceled)

33. The disinfection system of claim 29 wherein:
the determination of the intensities for the respective light sources includes determining a high intensity for light sources that do not impinge on an occupant as indicated by the occupancy map;
wherein the high intensity exceeds an intensity that would produce a dose exceeding an actinic dose limit if received over a design-basis dose time period over which the actinic dose limit is defined.

34. (canceled)
